(19) United States
(12) Patent Application Publication
(10) Pub. No.: US 2017/0199179 A1
(43) Pub. Date: Jul. 13, 2017

(54) PD-IR NANOPARTICLES USED AS PEROXIDASE MIMICS

(71) Applicant: Michigan Technological University, Houghton, MI (US)

(72) Inventor: Xiaohu Xia, Hancock, MI (US)

(21) Appl. No.: 15/376,513

(22) Filed: Dec. 12, 2016

Related U.S. Application Data
(60) Provisional application No. 62/266,201, filed on Dec. 11, 2015.

(51) Int. Cl. G01N 33/53; G01N 33/52

(52) U.S. Cl. CPC G01N 33/5308; G01N 33/526; G01N 33/525

(57) ABSTRACT

Described herein are methods of catalyzing oxidation reactions by an oxidizing agent (e.g., hydrogen peroxide). The methods include contacting a substrate with an oxidizing agent and a nanoparticle in a reaction mixture, wherein the nanoparticle comprises a Pd core at least partially surrounded by a coating comprising Ir. In addition, disclosed herein are kits for catalyzing the oxidation of a substrate by an oxidizing agent. The kits include a substrate, an oxidizing agent, and a nanoparticle, wherein the nanoparticle comprises a Pd core at least partially surrounded by a coating comprising Ir. The kits also include instructions describing how to contact the substrate, the oxidizing agent and the nanoparticle so as to catalyze the oxidation of the substrate.

### CROSS-REFERENCE TO RELATED APPLICATIONS

This application claims priority to U.S. Provisional Application No. 62/266,201 filed on Dec. 11, 2015, which is incorporated fully herein by reference.

### BACKGROUND

Peroxidase mimics with dimensions on the nanoscale have received great interest as emerging artificial enzymes for biomedicine and environmental protection. While a variety of peroxidase mimics have been actively developed recently, limited progress has been made toward improving their catalytic efficiency. Accordingly, there is a need for nanoscale peroxidase mimics that have improved catalytic efficiency.

### SUMMARY

In some aspects, the present disclosure provides methods of catalyzing the oxidation of a substrate by an oxidizing agent, the methods comprising contacting the substrate with the oxidizing agent and a nanoparticle in a reaction mixture, wherein the nanoparticle comprises a Pd core at least partially surrounded by a coating comprising Ir.

The methods may further include wherein the nanoparticle is coupled to a binding agent adapted to bind to a target analyte, and wherein the methods further comprise contacting the nanoparticle and the binding agent with the target analyte to form a complex comprising the nanoparticle, the binding agent and the target analyte, and contacting the complex with the substrate and the oxidizing agent in the reaction mixture.

In addition, the methods may include wherein the substrate is coupled to a binding agent adapted to bind to a target analyte, and wherein the methods further comprise contacting the substrate and the binding agent with the target analyte to form a complex comprising the substrate, the binding agent and the target analyte, and contacting the complex with the nanoparticle and the oxidizing agent in the reaction mixture.

In other aspects, the present disclosure provides kits for catalyzing the oxidation of a substrate by an oxidizing agent, comprising the substrate, the oxidizing agent; and a nanoparticle, wherein the nanoparticle comprises a Pd core at least partially surrounded by a coating comprising Ir; and instructions describing how to contact the substrate, the oxidizing agent and the nanoparticle so as to catalyze the oxidation of the substrate.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 is a schematic showing the growth of Ir on Pd cube seeds.

FIG. 2 is a schematic showing the growth of Ir on Pd truncated octahedral seeds.

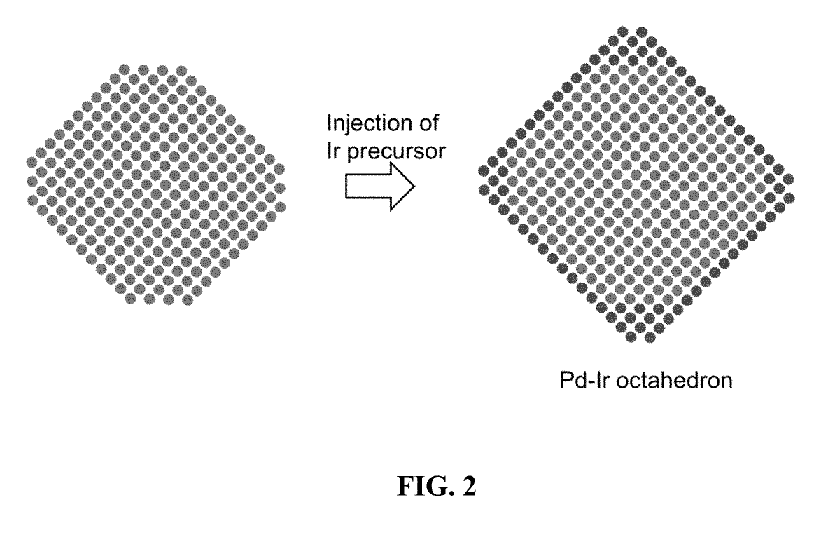

FIG. 3 is a transmission electron microscopy (TEM) image of Pd cubes with average edge lengths of approximately 7.2 nm that served as cubic seeds.

FIG. 4 is a TEM image of Pd cubes with average edge lengths of approximately 18 nm that served as cubic seeds.

FIG. 5 is a TEM image of Pd cubes with average edge lengths of approximately 51 nm that served as cubic seeds.

FIG. 6 is (a) a TEM image of Pd—Ir cubes having average edge lengths of approximately 19.2 nm; and (b) a high-angle annular dark-field imaging-scanning transmission electron microscopy (HAADF-STEM) image of a Pd—Ir nanoparticle.

FIG. 7 is (a) an atomic-resolution HAADF-STEM taken from the corner region of a Pd—Ir nanoparticle (NP); and (b) a line-scan energy-dispersive X-ray (EDX) spectra of elemental Pd and Ir recorded from an individual Pd—Ir NP nanoparticle (inset: along an edge-to-edge direction as indicated by the arrow).

FIG. 8 is a TEM image of Pd octahedral nanoparticles with an average edge length of 6 nm that served as the seeds for the growth of Ir. The inset shows a TEM image taken from the same sample at a higher magnification.

FIG. 9 is a TEM image of Pd truncated octahedral nanoparticles with an overall spherical shape and an average size of about 5.6 nm. The inset shows a high resolution TEM (HRTEM) image recorded from an individual particle.

FIG. 10 is (a) a TEM image of Pd—Ir core-shell octahedral nanoparticles. (The inset shows a TEM image taken from the same sample at a higher magnification and a 2D schematic model.); and (b) a HRTEM image of an individual Pd—Ir octahedron nanoparticle.

FIG. 11 is a HAADF-STEM image of an individual Pd—Ir octahedron nanoparticle together with EDX maps.

FIG. 12 is (a) an image taken at reaction time t=5 minutes, showing the capability of Pd—Ir cubes in catalyzing the oxidations of various peroxidase substrates; and (b) a schematic showing the oxidation of 3,3′,5,5′-Tetramethylbenzidine (TMB) by H₂O₂ to form blue-colored products.

FIG. 13 is (a) an ultraviolet (UV)-visible spectrum taken from sample #4 of FIG. 12(a); and (b) time- and particle concentration dependent absorbance at 653 nm measured from the reaction solutions.

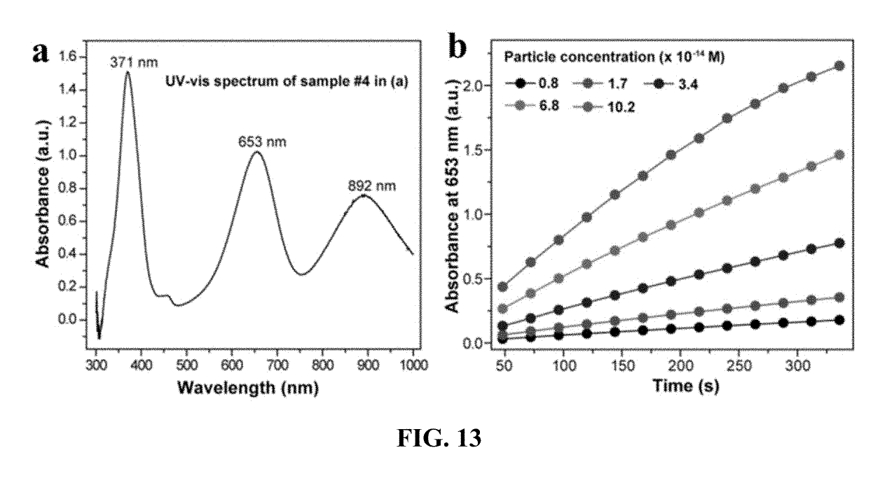

FIG. 14 shows a steady-state kinetic analysis towards TMB, and includes: (a) a plot of v against TMB concentration, in which H₂O₂ concentration was fixed at 2.0 M; (b) a double-reciprocal plot generated from (a); and (c) double-reciprocal plots at different H₂O₂ concentrations.

FIG. 15 shows steady-state kinetic analysis towards H₂O₂, and includes: (a) a plot of v against H₂O₂ concentration, in which TMB concentration was fixed at 0.8 mM; (b) a double-reciprocal plot generated from (a); and (c) double-reciprocal plots at different TMB concentrations.

FIG. 16 is a bar chart showing the effect of Ir content on the catalytic efficiency of Pd—Ir cubes.

FIG. 17 is a schematic showing the use of the Pd—Ir nanoparticles in an enzyme-linked immunosorbent assay (ELISA).

FIG. 18 is a photograph showing the detection of prostate-specific antigen (PSA) standards with Pd—Ir ELISA (top row), and horseradish peroxidase (HRP) ELISA (bottom row).

FIG. 19 is a plot of corresponding calibration curves of the detection results shown in FIG. 18. Error bars indicate the standard deviations of six independent measurements (inset shows the linear range region of a Pd—Ir ELISA assay).

FIG. 20 is a plot showing the detection of hydroxyl radical (OOH) generated from H₂O₂ using terephthalic acid (TA) as a probe.

FIG. 21 depicts an analysis of the PEGylation of Pd—Ir cubes and the effect of PEG chain coverage on catalytic activity, and includes: (a) a schematic showing the PEGylation process and the method for quantifying PEG chains on the surface of Pd—Ir cubes, and (b) a plot showing the relative catalytic activities (e.g., absorbance at 653 nm of reaction solutions at reaction time t =5 min) of PEGylated Pd—Ir nanoparticles. Datum at each point represents the average of three independent measurements.

FIG. 22 is a TEM image of Pd—Ir sphere nanoparticles.

FIG. 23 is a TEM image of Pd—Ir octahedral nanoparticles.

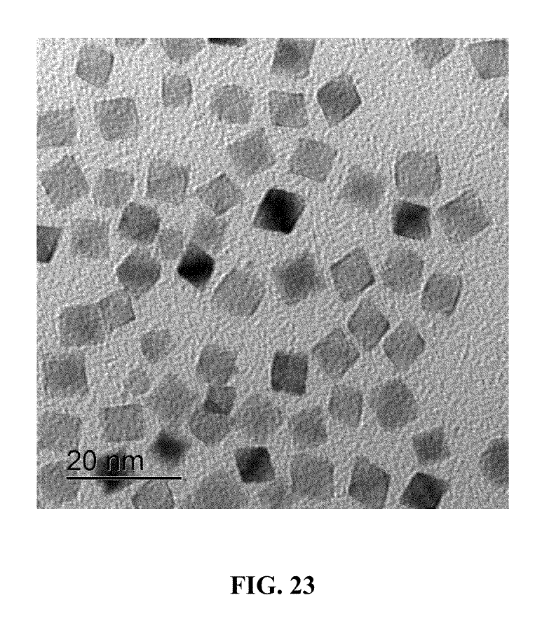

FIG. 24 is a TEM image of Pd—Ir cube nanoparticles having an average length of about 8.4 nm.

FIG. 25 is a TEM image of image of Pd—Ir cube nanoparticles having an average length of about 52.2 nm.

FIG. 26 is a structural and composition analysis of Pd—Ir octahedral nanoparticles, and includes: (a) & (b) TEM images of Pd—Ir octahedra nanoparticles with an average size of 6.1 nm (the inset of (b) is a 2D schematic model); (c) a HRTEM image of an individual Pd—Ir nanoparticle; and (d) a HAADF-STEM image and EDX mapping images of an individual Pd—Ir nanoparticle.

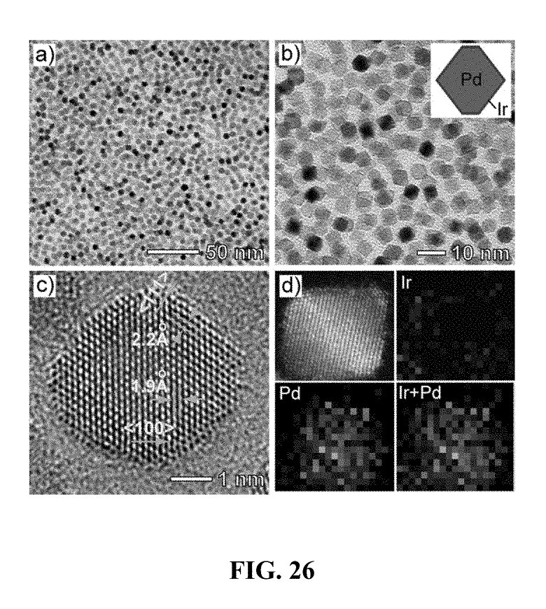

FIG. 27 is an analysis of the effect of (a) pH, (b) temperature, (c) TMB concentration, and (d) H₂O₂ concentration on the catalytic activity of 19.2 nm Pd—Ir nanocubes. The maximum absorbance at 653 nm of reaction solutions at t=5 min in each plot (a-d) was set as 100%.

FIG. 28 shows a stability test of 19.2 nm Pd—Ir nanocubes in catalyzing the oxidation of TMB by H₂O₂, and includes: (a) a plot showing the activity of Pd—Ir nanocubes as a function of pH; and (b) a plot showing the activity of Pd—Ir nanocubes as a function of temperature.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The conjunctive term “or” includes any and all combinations of one or more listed elements associated by the conjunctive term. For example, the phrase “an apparatus comprising A or B” may refer to an apparatus including A where B is not present, an apparatus including B where A is not present, or an apparatus where both A and B are present. The phrases “at least one of A, B, . . . and N” or “at least one of A, B, . . . N, or combinations thereof” are defined in the broadest sense to mean one or more elements selected from the group comprising A, B, . . . and N, that is to say, any combination of one or more of the elements A, B, . . . or N including any one element alone or in combination with one or more of the other elements which may also include, in combination, additional elements not listed.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

### 2. Methods of Catalyzing Oxidation

In some aspects, this disclosure provides methods of catalyzing the oxidation of a substrate by an oxidizing agent. The methods may include contacting the substrate with the oxidizing agent and a nanoparticle in a reaction mixture, wherein the nanoparticle comprises a Pd core at least partially surrounded by a coating comprising Ir. The methods may significantly improve the kinetics of oxidation (compared to known catalysts for the same oxidation, e.g., horseradish peroxidase in H₂O₂ oxidation) by mediating the oxidation through the nanoparticle comprising a Pd core and a coating at least partially surrounding the Pd core, wherein the coating includes Ir (i.e., a Pd—Ir nanoparticle).

The efficiency of the catalyst in the methods of the present disclosure may be affected by parameters such as pH of the reaction mixture, temperature of the reaction mixture, the amount of Ir present in the coating of the nanoparticle, the molecular weight of the capping agent used to synthesize the nanoparticle, and/or further modifications of the nanoparticle (e.g., PEGylation).

Depending on the application and the parameters of the reaction, the method may be performed for a specified amount of time. The method may be performed from seconds to hours depending on the application. For example, the method may be performed for 5 seconds, 10 seconds, 30 seconds, 1 minute, 5 minutes, 10 minutes, 30 minutes, 1 hour, or 5 hours.

### A. Reaction Mixture

The methods disclosed herein may include a reaction mixture. The reaction mixture may be a solution and/or suspension. In addition, the reaction mixture may be an aqueous solution and/or suspension. The reaction mixture may include a substrate, an oxidizing agent (e.g., hydrogen peroxide), and a nanoparticle comprising a Pd core at least partially surrounded by a coating that includes Ir.

The temperature at which the oxidation reaction takes place may affect the catalytic efficiency of the Pd—Ir nanoparticle, or in other words, the temperature of the reaction mixture may affect the catalytic efficiency of the Pd—Ir nanoparticle. The reaction mixture may have a temperature of from about 10° C. to about 50° C. or from about 10° C. to about 37° C. For example, the reaction mixture may have a temperature of about 10° C., about 15° C., about 20° C., about 25° C., about 30° C., about 35° C., about 40° C., about 45° C., or about 10° C.

The pH at which the oxidation reaction takes place may affect the catalytic efficiency of the Pd—Ir nanoparticle, or in other words, the pH of the reaction mixture may affect the catalytic efficiency of the Pd—Ir nanoparticle. The reaction mixture may have a pH of from about 2 to about 8. For example, the reaction mixture may have a pH of about 2, about 3, about 4, about 5, about 6, about 7, or about 8. In some embodiments, the reaction mixture may have a pH of greater than about 1, greater than about 2, greater than about 3, or greater than about 4.

i. Substrate

The reaction mixture may include a substrate that can be oxidized by an oxidizing agent (e.g., hydrogen peroxide). When combined with the oxidizing agent and the Pd—Ir nanoparticle, the substrate may be oxidized, wherein the Pd—Ir nanoparticle catalyzes the oxidation reaction. Oxidation of the substrate may yield a characteristic change to the substrate. For example, following oxidation, the substrate may be detectable through spectrophotometric methods. In some embodiments, the substrate may be chromogenic, chemiluminescent, fluorogenic, electrochemiluminescent or a combination thereof

Substrates suitable for use in the disclosed methods may include any substrate that is known to be oxidized by hydrogen peroxide in the presence of a peroxidase. Examples of suitable substrates may include (but are not limited to) 2,T-azino-bis(3-ethylbenzthiazoline-6-sulfonic acid) (ABTS), 3,3′,5,5′-tetramethylbenzidine (TMB), 3,3′,5,5′-tetramethylbenzidine sulfate (TMBS), o-phenylenediamine (OPD), o-phenylenediamine dihydrochloride (OPD-2HCl), diaminobenzidine (DAB), diaminobenzidine tetrahydrochloride (DAB-4HCl), 3-amino-9-ethylcarbazole (AEC), 10-acetyl-3,7-dihydroxyphenoxazine (Ampliflu Red), luminol, homovanillic acid, 5-aminosalicylic acid (5-ASA), and a combination thereof

In some embodiments, the substrate may be present in the reaction mixture at a concentration of from about 0.1 nM to about 10 M (e.g., from about 0.1 mM to about 1 M, or from about 0.6 mM to about 1.2 mM) although the substrate concentration is not limited to this range. For example, the substrate may be present in the reaction mixture at a concentration of about 0.1 nM, about 1 nM, about 0.1 μM, about 1 μM, about 0.05 mM, about 0.08 mM, about 0.1 mM, about 0.2 mM, about 0.4 mM, about 0.6 mM, 0.8 mM, about 1 mM, about 1.5 mM, about 2 mM, about 4 mM, about 6 mM, about 8 mM, about 10 mM, about 100 mM, about 1 M, or about 10 M.

In some embodiments, the substrate may be present in the reaction mixture at a concentration of greater than about 0.1 nM, greater than about 1 nM, greater than about 0.1 μM, greater than about 1 μM, greater than about 0.1 mM, or greater than about 1 mM.

In some embodiments, the substrate may be present in the reaction mixture at a concentration less than that of the oxidizing agent. For example, the substrate may be present in the reaction mixture at a concentration about 2× less than, about 5× less than, about 10× less than, about 100× less than, about 1,000× less than, about 10,000× less than, or about 100,000× less than the concentration of the oxidizing agent in the same reaction mixture.

In some embodiments, the substrate may be present in the reaction mixture at a concentration greater than that of the nanoparticle. For example, the substrate may be present in the reaction mixture at concentration about 1,000× greater than, about 10,000× greater than, about 100,000× greater than, about 1×10⁶× greater than, about 1×10⁸× greater than, about 1 x10¹⁰× greater than, about 1×10¹¹× greater than, or about 1×10¹²× greater than the concentration of the nanoparticle in the same reaction mixture.

ii. Oxidizing Agent

The reaction mixture may include an oxidizing agent, e.g., a substance that has the ability to oxidize other substances, thereby causing the oxidized substance to lose electrons. The oxidizing agent may include one or more of hydrogen peroxide, sodium perborate and sodium percabonate, and any other suitable oxidizing agent. When combined with the substrate and the Pd—Ir nanoparticle, the oxidizing agent may oxidize the substrate, wherein the Pd—Ir nanoparticle catalyzes the oxidation reaction. The oxidation may yield a characteristic change to the substrate. For example, following oxidation the substrate may be detectable through spectrophotometric methods.

The oxidizing agent may be present in the reaction mixture at a concentration of from about 0.1 M to about 20 M, such as from about 1 M to about 10 M or from about 1.5 M to about 7 M (although the oxidizing agent concentration is not limited to this range). For example, the oxidizing agent may be present in the reaction mixture at a concentration of about 0.1 M, about 0.2 M, about 0.4 M, about 0.8 M, about 1 M, about 1.5 M, about 2 M, about 2.5 M, about 3 M, about 3.5 M, about 4 M, about 4.5 M, about 5 M, about 5.5 M, about 6 M, about 6.5 M, about 7 M, about 7.5 M, about 8 M, about 8.5 M, about 9 M, about 9.5 M, about 10 M, about 11 M, about 12 M, about 13 M, about 14 M, about 15 M, about 16 M, about 17 M, about 18 M, about 19 M, or about 20 M. In some embodiments, the oxidizing agent may be hydrogen peroxide and may be present in the reaction mixture at a concentration of about 7 M.

In some embodiments, the oxidizing agent may be present in the reaction mixture at a concentration greater than that of the substrate. For example, the oxidizing agent may be present in the reaction mixture at a concentration about 2× greater than, about 5× greater than, about 10x greater than, about 100× greater than, about 1,000× greater than, about 10,000× greater than, or about 100,000× greater than the concentration of the substrate in the same reaction mixture.

In some embodiments, the oxidizing agent may be present in the reaction mixture at a concentration greater than that of the nanoparticle. For example, the oxidizing agent may be present in the reaction mixture at a concentration about 1,000× greater than, about 10,000× greater than, about 100,000× greater than, about 1×10⁶× greater than, about 1×10⁸× greater than, about 1×10¹⁰× greater than, about 1×10¹²× greater than, about 1×10¹⁴× greater than, or about 1×10¹⁶× greater than the concentration of the nanoparticle in the same reaction mixture.

iii. Nanoparticles

The reaction mixture may include and/or be contacted with a nanoparticle comprising a Pd core at least partially surrounded by a coating comprising Ir. The Pd core may act as a seed for the growth of the coating to form the nanoparticle, such that the seed is subsequently coated (at least partially) with a coating comprising Ir. The nanoparticle may further include a capping agent. In some embodiments, the nanoparticle may be modified with a variety of surface modifications, including (but not limited to) PEGylation of the nanoparticle surface.

The Pd—Ir nanoparticles disclosed herein have the ability to function as peroxidase mimics, as they can catalyze the oxidation of the substrate with the oxidizing agent (e.g., hydrogen peroxide). Unlike natural peroxidases, peroxidase mimics may be less vulnerable to denaturation and protease digestion and therefore may be significantly more stable. Further, the nanoparticles of the present disclosure may catalyze the oxidation reaction in an efficient manner. For example, the nanoparticle may have a k_{cat} toward the substrate of from about 1×10⁵ s⁻¹ to about 1×10¹⁰ s⁻¹, such as from about 2×10⁵ s⁻¹ to about 1×10⁸ s⁻¹, or from about 3×10⁵ s⁻¹ to about 5×10⁷ s⁻¹. The nanoparticle may have a k_{cat} toward the substrate of about 1×10⁵ s⁻¹, about 2×10⁵ s⁻¹, about 3×10⁵ s⁻¹ , about 3.9×10⁵ s⁻¹, about 4×10⁵ s⁻¹, about 6×10⁵ s⁻¹, about 8×10⁵ s⁻¹ , about 1×10⁶ s⁻¹, about 1.9×10⁶ s⁻¹, about 2×10⁶ s⁻¹, about 4×10⁶ s⁻¹, about 6×10⁶ s⁻about 8×10⁶ s⁻¹ about 1×10⁷ s⁻¹ about 1.2×10⁷ s⁻¹ about 2×10⁷ s⁻¹ about 4×10⁷ s⁻¹, about 6×10⁷ s⁻, about 8×10⁷ s⁻¹, about 1×10⁸ s⁻¹, about 5×10⁸ s⁻¹, about 1×10⁹ s⁻¹, about 5×10⁹ s⁻¹, or about 1×10¹⁰ s⁻¹.

In some embodiments, the nanoparticles may have a k_{cat} toward the substrate of greater than about 1×10⁵ s⁻¹, greater than about 3×10⁵ s⁻¹, greater than about 3.9×10⁵ s⁻¹, greater than about 4×10⁵ s⁻¹, greater than about 6×10⁵ s⁻¹, greater than about 8×10⁵ s⁻¹, greater than about 1×10⁶ s⁻¹, greater than about 1.9×10⁶ s⁻¹, greater than about 2×10⁶ s⁻¹, greater than about 4×10⁶ s⁻¹, greater than about 6×10⁶ s⁻¹, greater than about 8×10⁶ s⁻¹, or greater than about 1×10⁷ s⁻¹.

The nanoparticles may have a number of different shapes, which may be dependent on the shape of the Pd core. For example, the nanoparticle may have a shape selected from the group consisting of cubic, truncated cubic, octahedral, truncated octahedral, and spherical.

The nanoparticles of the present disclosure may have variable surface area and size, depending (in part) on the size and shape of the Pd core, the amount of Ir in the coating, and the type/amount of any capping agent. The average diameter of the nanoparticle may be measured by techniques known within the art such as, dynamic light scatter and electron microscopy. The nanoparticle may have an average diameter of from about 5 nm to about 200 nm, 8 nm to about 100 nm, or from about 8 nm to about 55 nm. For example, the nanoparticle may have an average diameter of about 5 nm, about 8 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 90 nm, about 100 nm, about 125 nm, about 150 nm, about 175 nm or about 200 nm.

The nanoparticle may also be described by its surface area, and this can be measured by similar techniques mentioned above and then surface area can be calculated per the shape of the nanoparticle. The nanoparticle may have an average surface area of from about 30 nm² to about 3×10⁵ nm², from about 100 nm² to about 6×10⁴ nm², or from about 400 nm² to about 2×10⁴ nm². For example, the nanoparticle may have an average surface area of about 30 nm², about 50 nm², about 100 nm², about 200 nm², about 400 nm², about 600 nm², about 800 nm², about 1×10³ nm², about 2×10³ nm², about 4×10³ nm², about 6×10³ nm², about 8×10³ nm², about 1×10⁴ nm², about 2×10⁴ nm², about 4×10³ nm⁴, about 6×10⁴ nm², about 8×10⁴ nm², about 1×10⁵ nm², or about 3×10⁵ nm².

In some embodiments, the nanoparticle may have a cube shape, and thus can be described by a dimension of edge length. A nanoparticle having a cube shape may have an average edge length of from about 1 nm to about 200 nm, from about 5 nm to about 100 nm, or from about 8.4 nm to about 52.2 nm. For example, a nanoparticle having a cube shape may have an average edge length of about 1 nm, about 5 nm, about 8.4 nm, about 10 nm, about 19.2 nm, about 20 nm, about 40 nm, about 50 nm, about 52.2 nm, about 75 nm, about 100 nm, about 150 nm, or about 200 nm.

The nanoparticle may include Pd and Ir at a molar ratio of from about PdIr_{0.01} to about PdIr_{0.3}, from about PdIr_{0.02}to about PdIr_{0.25}, or from about PdIr_{0.05} to about PdIr_{0.2}. For example, the nanoparticle may comprises Pd and Ir at a molar ratio of about PdIr_{0.01}, about PdIr_{0.015}, about PdIr_{0.02}, about PdIr_{0.025}, about PdIr_{0.03}, about PdIr_{0.035}, about PdIr_{0.04}, about PdIr_{0.045}, about PdIr_{0.05}, about PdIr_{0.055}, about PdIr_{0.06}, about PdIr_{0.065}, about PdIr_{0.07}, about PdIr_{0.075}, about PdIr_{0.08}, about PdIr_{0.085}, about PdIr_{0.09}, about PdIr_{0.1}, about PdIir_{0.12}, about PdIr_{0.14}, about PdIr_{0.16}, about PdIr_{0.18}, about PdIr_{0.2}, about PdIr_{0.22}, about PdIr_{0.24}, about PdIr_{0.26}, about PdIr_{0.28}, or about PdIr_{0.3}. In some embodiments, the nanoparticle includes Pd and Ir at a molar ratio of about PdIr_{0.062}.

The nanoparticle may be present in the reaction mixture as a plurality of nanoparticles. In some embodiments, the nanoparticle may be present in the reaction mixture at a concentration of from about 0.1×10⁻¹⁴ M to about 1 M (e.g., from about 0.5×10⁻¹⁴ M to about 1×10⁻⁶M, or from about 0.8×10⁻¹⁴ M to about 10.2×10⁻¹⁴ M), although the nanoparticle concentration is not limited to this range. For example, the nanoparticle may be present in the reaction mixture at a concentration of about 0.1×10⁻¹⁴ M, about 0.2×10−¹⁴M, about 0.3×10⁻¹⁴ M, about 0.4×10⁻¹⁴ M, about 0.5×10−¹⁴M, about 0.6×10⁻¹⁴M, about 0.7×10⁻¹⁴ M, about 0.8×10⁻¹⁴ M, about 0.9×10⁻¹⁴ M, about 1×10⁻¹⁴ M, about 2×10⁻¹⁴ M, about 4×10⁻¹⁴M, about 5×10⁻¹⁴ M, about 6×10⁻¹⁴M, about 8×10−¹⁴M, about 10×10⁻¹⁴M, about 10.2×10⁻¹⁴M, about 15×10⁻¹⁴M, about 20×10⁻¹⁴M, about 1×10⁻¹²M, about 1×10⁻¹M, about 1×10⁻⁸M, about 1×10⁻⁶M, about 1×10⁻⁴M, about 1×10 ⁻²M, or about 1 M. In some embodiments, the nanoparticle may be present in the reaction mixture at a concentration of greater than about 0.1×10⁻¹⁴ M, greater than about 0.5×10⁻¹⁴ M, greater than about 1×10⁻¹⁴ M, greater than about 5×10⁻¹⁴ M, or greater than about 10×10⁻¹⁴ M. In other embodiments the nanoparticle concentration may be present in the reaction mixture at a concentration of less than about 20×10¹⁴M, less than about 15×10⁻¹⁴ M, less than about 10×10⁻¹⁴ M, less than about 5×10⁻¹⁴ M, or less than about 1×10⁻¹⁴ M.

a. Pd Core

As described above, the Pd core may be a seed for providing the nanoparticle, and may have a number of different shapes. For example, the Pd core may have a shape selected from the group consisting of cubic, truncated cubic, octahedral, truncated octahedral, and spherical. The shape of the Pd core may influence the shape of the nanoparticle. The Pd core may have a smooth surface or an irregular (e.g., unsmooth) surface. In some embodiments, the Pd core may include {100} facets, {111} facets, or a combination thereof. In some embodiments, the Pd core may include {100}, {110} and {111} facets together. In some embodiments, the Pd core may include high-index facets, like {730}, {420}, or {311}. In some embodiments, the Pd core may have a single crystal structure or a multiple crystal structure.

The Pd core may have a variable surface area and size. The average diameter of the Pd core may be measured by techniques known within the art such as, dynamic light scatter and electron microscopy. The Pd core may have an average diameter of from about 5 nm to about 200 nm, such as from about 8 nm to about 100 nm, or from about 8 nm to about 55 nm. For example, the Pd core may have an average diameter of about 5 nm, about 8 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 90 nm, about 100 nm, about 125 nm, about 150 nm, about 175 nm or about 200 nm.

The Pd core may also be described by its surface area, and this can be measured by similar techniques mentioned above and then the surface area may be calculated per the shape of the Pd core. Further, this is not to be confused with the surface area of the nanoparticle or that the Pd core surface area would be considered the surface of the nanoparticle, but rather as a way of describing the size of the Pd core. The Pd core may have an average surface area of from about 25 nm² to about 2.5×10⁵ nm², such as from about 100 nm² to about 5×10⁴ nm², or from about 300 nm² to about 1.6×10⁴ nm². For example, the Pd core may have an average surface area of about 25 nm², about 50 nm², about 100 nm², about 200 nm², about 400 nm², about 600 nm², about 800 nm², about 1×10³ nm², about 2×10³ nm², about 4×10³ nm², about 6×10³ nm², about 8×10³ nm², about 1×10⁴ nm², about 1.6×10⁴ nm², about 2×10⁴ nm², about 4×10³ nm⁴, about 5×10⁴ nm², about 1×10⁵ nm², or about 2.5×10⁵ nm².

In some embodiments, the Pd core may have a cube shape, and thus can be described by a dimension of edge length. A Pd core having a cube shape may have an average edge length of from about 1 nm to about 200 nm, such as from about 5 nm to about 100 nm, or from about 7.2 nm to about 51 nm. For example, a Pd core having a cube shape may have an average edge length of about 1 nm, about 5 nm, about 7.2 nm, about 10 nm, about 18 nm, about 20 nm, about 40 nm, about 50 nm, about 51 nm, about 75 nm, about 100 nm, about 150 nm, or about 200 nm.

b. Capping Agent

The nanoparticle may comprise a capping agent. The capping agent may be used to stabilize the Pd cores and Ir coatings on said core. For example, the capping agent may be used to decrease or prevent aggregation of nanoparticles in a suspension. The capping agent may be present as part of the coating, on the surface of the nanoparticle, or a combination thereof.

In some embodiments, the capping agent may be polyvinylpyrrolidone. Polyvinylpyrrolidone may have a molecular weight of from about 10,000 g/mol to about 360,000 g/mol. For example, polyvinylpyrrolidone may have a molecular weight of about 10,000 g/mol, about 55,000 g/mol, about 100,000 g/mol, about 150,000 g/mol, about 200,000 g/mol, about 250,000 g/mol, about 300,000 g/mol, or about 360,000 g/mol.

c. Coating

The nanoparticles of the present disclosure comprise a coating that at least partially surrounds the Pd core and includes Ir. The coating may further comprise a capping agent, as discussed above. The coating may include Ir in a manner wherein the Ir has an epitaxial relationship with the Pd core. In some embodiments, the coating may include Ir {100} facets, Ir {111} facets, or a combination thereof. In some embodiments, the coating consists essentially of Ir. In some embodiments, the coating consists essentially of Ir and the capping agent.

The coating may be applied to the Pd core in a manner which can control the topography of the coating. For example, the coating may cover the core in a heterogeneous (e.g., uneven) manner, e.g., through 3-dimensional island growth. In other embodiments, the coating may cover the core in a homogenous manner, resulting in a smoother surface (compared to the aforementioned heterogeneous surface). The coating may cover a certain amount of surface area of the Pd core. For instance, the coating may cover greater than about 75% of the surface area of the Pd core, greater than about 80% of the surface area of the Pd core, greater than about 85% of the surface area of the Pd core, greater than about 90% of the surface area of the Pd core, greater than about 95% of the surface area of the Pd core, or greater than about 99% of the surface area of the Pd core. In some embodiments, the coating covers about 100% of the surface area of the Pd core.

The coating may have a thickness of from about 1 atomic layer to about 3 atomic layers (wherein the atom refers to Ir). For example, the coating may have, on average, a thickness of 1 atomic layer, 2 atomic layers or 3 atomic layers. In some embodiments, the coating may have an average thickness of from about 0.2 nm to about 2 nm, from about 0.5 nm to about 1.5 nm, or from about 0.6 nm to about 1.2 nm. For example, the coating may have an average thickness of about 0.2 nm, about 0.3 nm, about 0.4 nm, about 0.5 nm, about 0.6 nm, about 0.7 nm, about 0.8 nm, about 0.9 nm, about 1 nm, about 1.2 nm, about 1.4 nm, about 1.6 nm, about 1.8 nm, or about 2 nm. In some embodiments, the coating may have an average thickness of about 0.6 nm.

d. Binding Agent

The nanoparticles of the present disclosure may further comprise a binding agent adapted to bind specifically to a defined target. In some embodiments, the binding agent may be a peptide, an antibody (or fragment thereof), or a nucleic acid (e.g., an aptamer). In some embodiments, the peptide may be an antigen for a target antibody.

The binding agent may be bound to the nanoparticle through a linker, such as a synthetic linker. The linker may covalently bind to both the coating of the nanoparticle and the binding agent, thereby the linker may be a heterobifunctional linker. The linker may have a variable length, which may provide the binding agent more flexibility in binding to its target molecule. In some embodiments, the binding agent may be directly bound to the coating.

The linker may be any suitable oligomer or polymer that can be bound covalently to the coating present on the Pd core. For example, the synthetic linker may be bound to Ir in the coating via a thiol and then bound to the binding agent through functional groups including (but not limited to) carboxy (COOH) and amine (NH₂). In some embodiments, the linker may be a thiolated heterobifunctional polyethylene glycol (PEG), which binds to the binding agent through a carboxy or amine. In some embodiments, the molecular weight of the PEG is 3,400 g/mol.

The synthetic linker may be present on the surface of the nanoparticle coating at from about 5 linker molecules to about 10,000 linker molecules. Accordingly, the binding agent may be present on the nanoparticle coating (bound through the synthetic linker) at from about 5 linker molecules to about 10,000 linker molecules.

### 3. Uses of Methods

The disclosed methods may be useful for a number of different applications including, but not limited to, life science research, biomedical, agricultural, environmental, food and beverage, forensic, cosmetic, therapeutic, and diagnostic applications. For example, the disclosed methods may be used in colorimetric immunoassays, fluorescent immunoassays, chemiluminescent immunoassays and immunohistochemistry. These methods may include, but are not limited to ELISA assays, Western Blot assays and the like. The methods also may be used in the treatment of industrial wastewater because the disclosed methods may promote the conversion of toxic pollutants, such as phenols into harmless substances.

In some of the methods disclosed herein, the substrate or nanoparticle may be coupled to a binding agent adapted to specifically bind a target analyte (e.g., an antibody or other ligand specific for the target analyte) to form a complex. The substrate or nanoparticle may be coupled to the binding agent via a linker, such as a polymeric linker. In some embodiments the linker may be synthetic. In embodiments where the binding agent is coupled to the substrate, the complex formed between the target analyte, the substrate and the binding agent may then be contacted with the nanoparticle and the oxidizing agent, whereupon the nanoparticle catalyzes the oxidation of the substrate to form a product. Similarly, in embodiments where the binding agent is coupled to the nanoparticle, the complex formed between the target analyte, the nanoparticle and the binding agent may then be contacted with the substrate and the oxidizing agent, whereupon the nanoparticle catalyzes the oxidation of the substrate to form a product. For these assays, any difference in the detectable properties of the substrate and the product will allow for detection and/or quantification of the reaction that leads to the formation of product according to known methods, thereby allowing for the detection and/or quantification of the target analyte.

### 4. Kits

Also provided herein are kits, which may be used for any of the above-mentioned uses. For example, the present disclosure provides kits for assaying test samples for the presence of a target analyte. Kits according to the present disclosure include one or more reagents useful for practicing one or more assays (e.g., colorimetric immunoassays, fluorescent immunoassays, chemiluminescent immunoassays, immunohistochemistry, etc.) that include the disclosed methods. A kit may generally include a package with one or more containers holding the reagents, as one or more separate compositions or, optionally, as admixture where the compatibility of the reagents will allow. The kit may also include other material(s), which may be desirable from a user standpoint, such as a buffer(s), a diluent(s), a standard(s), and/or any other material useful in sample processing, washing, or conducting any other step of the assay.

The substrate, oxidizing agent and nanoparticle of the disclosed methods may be present within the kit as a number of different forms and packaging. For example, kits may include the substrate, the oxidizing agent and the nanoparticle all within separate containers. In some embodiments, the substrate and oxidizing agent are present within the kit as solutions and/or solids that need to be dissolved. In some embodiments, the nanoparticle is present within the kit as a suspension and/or solid that needs to be suspended. In some embodiments, the nanoparticle or the substrate may be coupled to a binding agent, as discussed above.

The kit may comprise instructions for using the compositions. Instructions included in kits may be affixed to packaging material or may be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### 5. Examples

Chemicals and Materials for the Examples

Sodium hexachloroiridate(III) hydrate (Na₃IrCl₆ 3 xH₂O, MW=473.9), sodium tetrachloropalladate(II) (Na₂PdCl₄, 98%), potassium bromide (KBr, ≧99%), L-ascorbic acid (AA, ≧99%), poly(vinylpyrrolidone) (PVP, MW≈55 000), TMB, >99%, DAB, ≧99%, OPD, ≧98%, hydrogen peroxide solution (30 wt % in H₂O), acetic acid (HOAc, ≧99.7%), sodium acetate (NaOAc, ≧99%), PSA, ≧99%, Tween 20, bovine serum albumin (BSA, ≧98%), N-hydroxysulfosuccinimide sodium salt (NHS, ≧98%), terephthalic acid (TA, ≧98%), N-ethyl-NO-(3-(dimethylamino)propyl)carbodiimide hydrochloride (EDC, ≧99%), sodium chloride (NaCl, ≧99.5%), potassium chloride (KCl, ≧99%), sodium phosphate dibasic (Na₂HPO₄, ≧99%), potassium phosphate monobasic (KH₂PO₄, >99%), Tris base(≧99.9%), sodium azide (NaN₃, ≧99.5%), citric acid (99%), ethylenediaminetetraacetic acid (EDTA, ≧99%), 5,50-dithiobis-(2-nitrobenzoic acid) (DTNB, ≧98%), and sulfuric acid (H₂SO₄, ≧98%) were all obtained from Sigma-Aldrich. Ethylene glycol (EG) was obtained from J. T. Baker, and HS-PEG₃₄₀₀-COOH(MW≈3400) and HS-PEG₃₄₀₀-NH₂ (MW≈3400) were obtained from Laysan Bio, Inc. Mouse anti-PSA monoclonal antibody (mouse anti-PSA mAb) and rabbit anti-PSA polyclonal antibody (rabbit anti-PSA pAb) were obtained from Abcam plc. Goat anti-mouse IgG and HRP-goat anti-mouse IgG conjugate were obtained from Thermo Fisher Scientific, Inc. Microtiter plates (96-well, polystryene, clear, flat bottom) were obtained from Corning Inc. All aqueous solutions were prepared using deionized (DI) water with a resistivity of 18.0 MΩ·cm.

The UV-vis spectra were recorded using an Agilent Cary 60 UV-vis spectrophotometer. TEM images were taken using a JEOL JEM-2010 microscope operated at 200 kV. HAADF-STEM and EDX analyses were performed using a JEOL ARM200F with STEM Cs corrector operated at 200 kV. The concentrations of Pd and Ir ions were determined using ICPAES (PerkinElmer Optima 7000DV), which could be converted to the particle concentration of Pd and Pd—Ir cubes once the particle size had been resolved by TEM imaging. The absorbance of samples in microtiter plates was read using a PerkinElmer Victor 3 1420 multilabel plate reader. Microtiter plates were shaken using a Corning LSE digital microplate shaker. pH values of buffer solutions were measured using an Oakton pH 700 benchtop meter. Photographs of samples in tubes and microplates were taken using a Canon EOS Rebel T5 digital camera. Fluorescence was measured using a Horiba Fluoromax-4 spectrofluorometer.

### Example 1

### Preparation and Characterization of Pd—Ir Nanoparticles

The method for generating the Pd—Ir nanoparticles with core-shell structure includes two steps: (a) synthesis of Pd seeds; (b) Growth of Ir on Pd seeds.

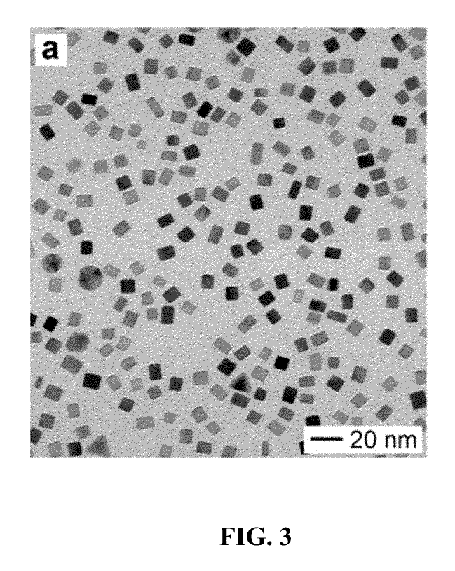

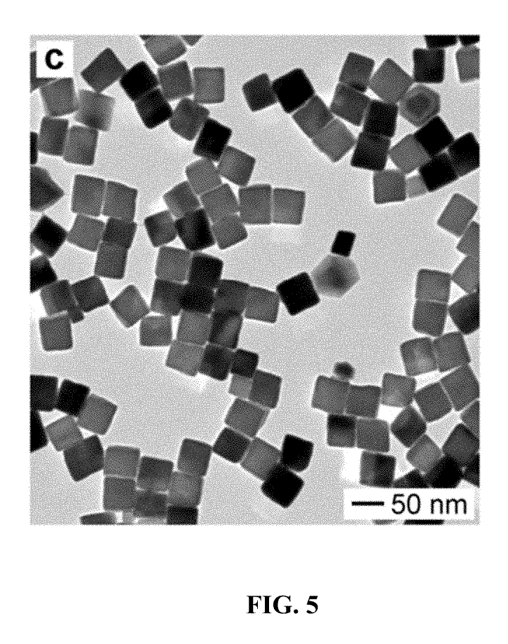

Pd Seed Growth. Step of (a), wherein the seed has cubic or truncated cubic structure. (1) As for the cubic seeds with an edge length of 7.2 nm (FIG. 3), 8.0 mL of an aqueous solution containing 105 mg of PVP, 60 mg of AA, and 8 mg of KBr and 180 mg of KCl was hosted in a vial and preheated at 80 ° C. in an oil bath under magnetic stirring for 10 min. Subsequently, 3.0 mL of a Na₂PdCl₄ aqueous solution (19 mg/mL) was added with a pipette. After the vial had been capped, the reaction was allowed to continue at 80 ° C. for 3 h. (2) As for the cubic seeds with an edge length of 18 nm, 8.0 mL of an aqueous solution containing 105 mg of PVP, 60 mg of AA, and 600 mg of KBr was hosted in a vial and preheated at 80° C. in an oil bath under magnetic stirring for 10 min. Subsequently, 3.0 mL of a Na₂PdCl₄ aqueous solution (19 mg/mL) was added with a pipette. After the vial had been capped, the reaction was allowed to continue at 80° C. for 3 h. (3) As for the cubic seeds with an edge length of 51 nm (FIG. 5), 12.0 mL of an aqueous solution containing 150 mg of PVP, 90 mg of AA, 450 mg of KBr, and 0.45 mL 18 nm Pd cubes (using as seeds, ˜4 mg/mL in Pd element) was hosted in a vial and preheated at 80 ° C. in an oil bath under magnetic stirring for 10 min. Subsequently, 5.0 mL of a Na₂PdC1₄ aqueous solution (19 mg/mL) was added with a pipette. After the vial had been capped, the reaction was allowed to continue at 80° C. for 24 h.

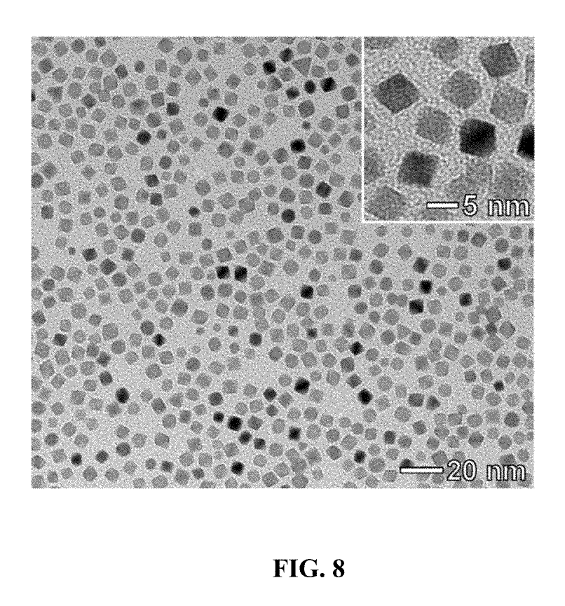

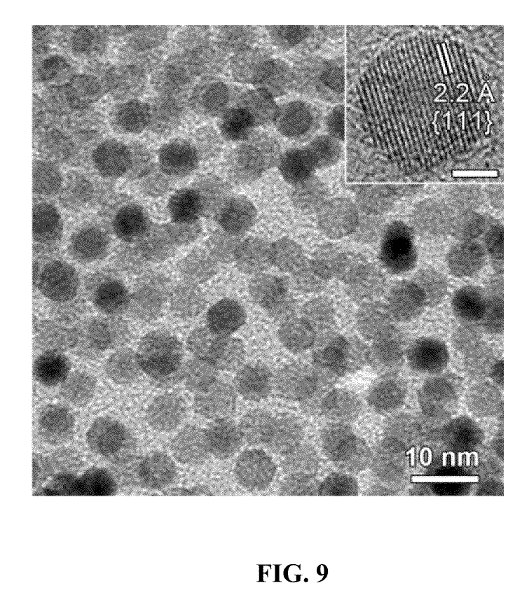

The step of (a), wherein the seed has an octahedral or truncated octahedral structure. (1) as for the truncated octahedral with a size (e.g., average diameter) of 5.6 nm (FIG. 9), 2 mL of an EG solution containing 100 mg of PVP was hosted in a glass vial and preheated to 160° C. in an oil bath under magnetic stirring for 10 min. Then, 1 mL of an EG solution containing 15.5 mg of Na₂PdCl₄ was quickly injected into the reaction solution using a pipette. The reaction was allowed to continue for 3 h. (2) As for the octahedral seeds with size of 6 nm (FIG. 8), 8 mL of water/ethanol mixture solution (5 mL water and 3 mL ethanol) containing 105 mg of PVP and 180 mg of CA was hosted in a vial and preheated to 80° C. in an oil bath under magnetic stirring for 10 min. Then, 3.0 mL of an aqueous solution containing 57 mg of Na₂PdCl₄ was added with a pipette. The reaction was allowed to continue at 80° C. for 3 h. (3) As for the octahedral seeds with size of 14 nm, 21 nm and 37 nm. 8 mL of water/ethanol mixture solution (5 mL water and 3 mL ethanol) containing 105 mg of PVP and 180 mg of CA was hosted in a vial and preheated to 80° C. in an oil bath under magnetic stirring for 10 min. Then, 3.0 mL of an aqueous solution containing 57 mg of Na₂PdCl₄ was added with a pipette. The reaction was allowed to continue at 80° C. for 3 h. In a standard procedure, 3 mL of aqueous Na2PdC14 solution (32 mM) was introduced into 8 mL of an aqueous solution containing 105 mg PVP, 100 μL HCHO, and 0.3 mL of an aqueous suspension (1.8 mg mL-1 in concentration) of Pd cubic seeds 18 nm in edge length, which had been heated at 60° C. for 5 min under magnetic stirring in a capped vial. Different from the Ag system, PVP seems to show negligible selectivity in binding to the {100} and {111} facets of Pd. As a result, the cubic seeds were forced by thermodynamic control to evolve into Pd polyhedrons with increasing presence of {111} facets as the amount of Na₂PdCl₄ was increased at a fixed amount of Pd seeds. The addition of Na₂PdCl₄ in the following amounts gave truncated cubes (5.8 mg), cuboctahedrons (8.7 mg), truncated octahedrons (17.4 mg), and octahedrons (29.0 mg), respectively. Each reaction was allowed to proceed at 60° C. for 3 h.

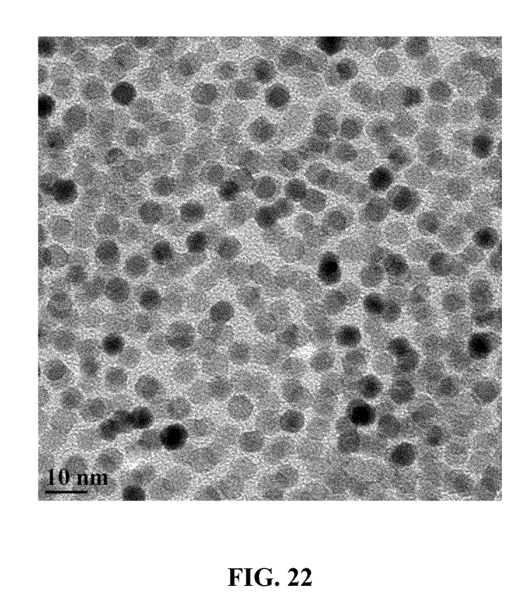

The step of (a), wherein the seed has a spherical structure having a diameter of 5 nm (FIG. 22). To obtain this seed, 2 mL of an EG solution containing 30 mg of PVP was hosted in a vial and preheated to 160° C. in an oil bath under magnetic stirring for 10 min. Then, 1.5 mL of an EG solution containing 16 mg of Na₂PdCl₄ was quickly added to the reaction solution with a pipette. The reaction was allowed to continue at 160° C. for 3 h.

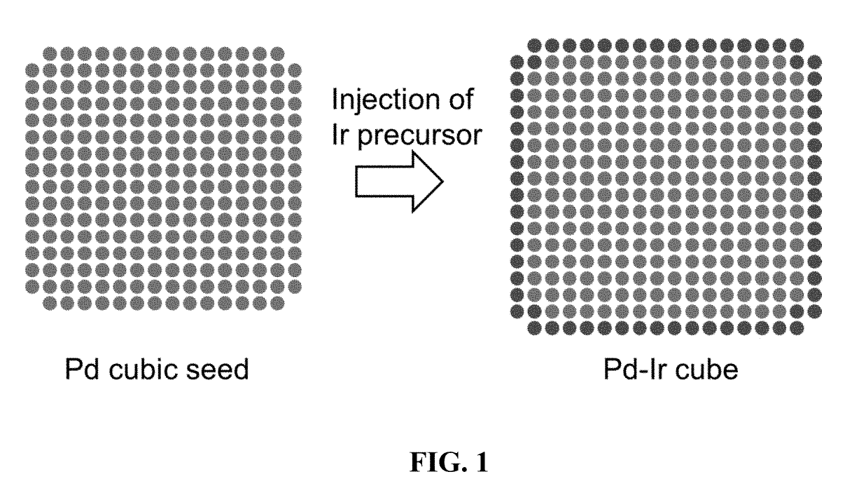

Ir Coating. The step of (b), wherein the Pd—Ir core-shell nanoparticles could be generated with cubic shape and different size based on the seeds (FIGS. 1 & 2). (1) As for the Pd—Ir cubes with length of 19.2 nm, 8 mL of an EG solution containing 100 mg of PVP and 50 mg of AA was hosted in a 25 mL three-neck flask and preheated to 200° C. in an oil bath under magnetic stirring for 10 min. Then, 1.0 mL of the 18 nm Pd cubic seeds (4 mg/mL in Pd element) was added to the flask using a pipette. After another 10 min, 4.0 mL of Na₃IrCl₆·xH₂O solution (0.8 mg/mL, in EG) was injected to the flask at a rate of 1.0 mL/h using a syringe pump. The reaction was allowed to proceed for an additional 5 min after the complete injection of the Na₃IrCl₆·×x₂O solution. The reaction was terminated by quickly placing the flask into an ice/water bath. (2) As for the Pd—Ir cubes with length of 8.4 nm, 8 mL of an EG solution containing 100 mg of PVP and 50 mg of AA was hosted in a 25 mL three-neck flask and preheated to 200 ° C. in an oil bath under magnetic stirring for 10 min. Then, 0.4 mL of the 7.2 nm Pd cubic seeds (4 mg/mL in Pd element) was added to the flask using a pipette. After another 10 min, 4.0 mL of Na₃IrCl₆·xH₂O solution (0.8 mg/mL, in EG) was injected to the flask at a rate of 1.0 mL/h using a syringe pump. The reaction was allowed to proceed for an additional 5 min after the complete injection of the Na₃IrCl₆·xH₂O solution. The reaction was terminated by quickly placing the flask into an ice/water bath. (3) As for the Pd—Ir cubes with length of 52.2 nm, 8 mL of an EG solution containing 100 mg of PVP and 50 mg of AA was hosted in a 25 mL three-neck flask and preheated to 200° C. in an oil bath under magnetic stirring for 10 min. Then, 0.6 mL of the 51 nm Pd cubic seeds (18 mg/mL in Pd element) was added to the flask using a pipette. After another 10 min, 4.0 mL of Na₃IrCl₆·xH₂O solution (0.8 mg/mL, in EG) was injected to the flask at a rate of 1.0 mL/h using a syringe pump. The reaction was allowed to proceed for an additional 5 min after the complete injection of the Na₃IrCl₆·xH₂O solution. The reaction was terminated by quickly placing the flask into an ice/water bath.

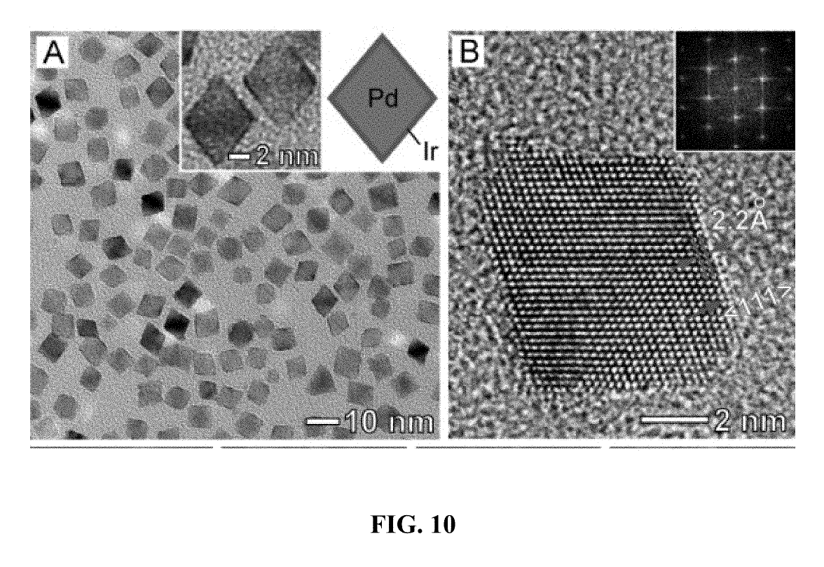

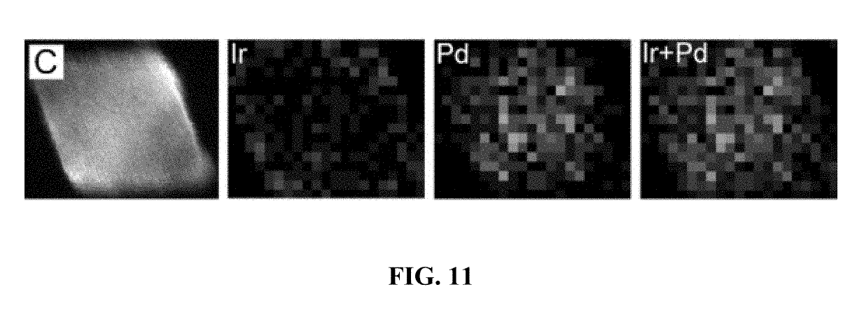

The step of (b), wherein the Pd—Ir core-shell nanoparticles could be generated with octahedral shape and different size based on the seeds (FIGS. 10, 11, 22 & 25). (1) As for the Pd—Ir octahedra with size of 6.8 nm, 8 mL of an EG solution containing 100 mg of PVP and 50 mg of AA was hosted in a 25 mL three-neck flask and preheated to 200° C. in an oil bath under magnetic stirring for 10 min. Then, 1.0 mL of the 5.6 nm truncated Pd cubic seeds (5.5 mg/mL in Pd element) was added to the flask using a pipette. After another 10 min, 2.0 mL of Na₃IrCl₆·xH₂O solution (1 mg/mL, in EG) was injected to the flask at a rate of 1.0 mL/h using a syringe pump. The reaction was allowed to proceed for an additional 5 min after the complete injection of the Na₃IrCl₆·xH₂O solution. The reaction was terminated by quickly placing the flask into an ice/water bath. (2) As for the Pd—Ir octahedra with size of 7.2 nm, 8 mL of an EG solution containing 100 mg of PVP and 50 mg of AA was hosted in a 25 mL three-neck flask and preheated to 200° C. in an oil bath under magnetic stirring for 10 min. Then, 1.0 mL of the 6 nm Pd octahedral seeds (4 mg/mL in Pd element) was added to the flask using a pipette. After another 10 min, 2.0 mL of Na₃IrCl₆·xH₂O solution (0.8 mg/mL, in EG) was injected to the flask at a rate of 1.0 mL/h using a syringe pump. The reaction was allowed to proceed for an additional 5 min after the complete injection of the Na₃IrCl₆·xH₂O solution. The reaction was terminated by quickly placing the flask into an ice/water bath.

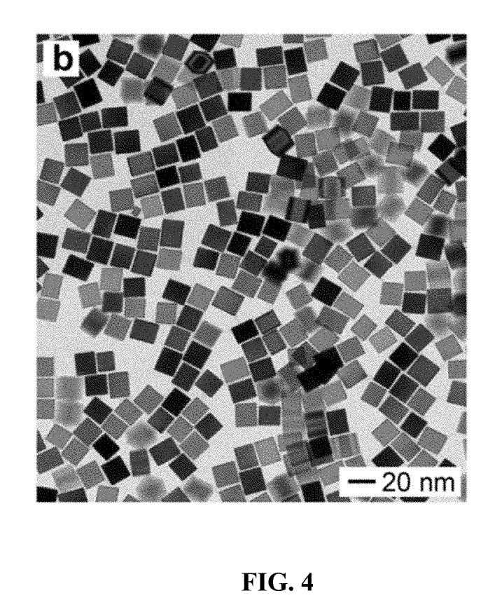

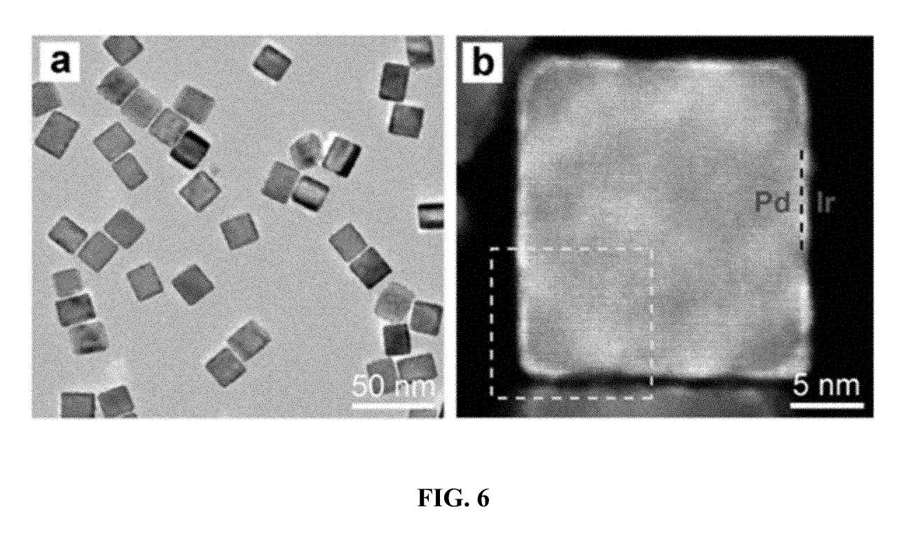

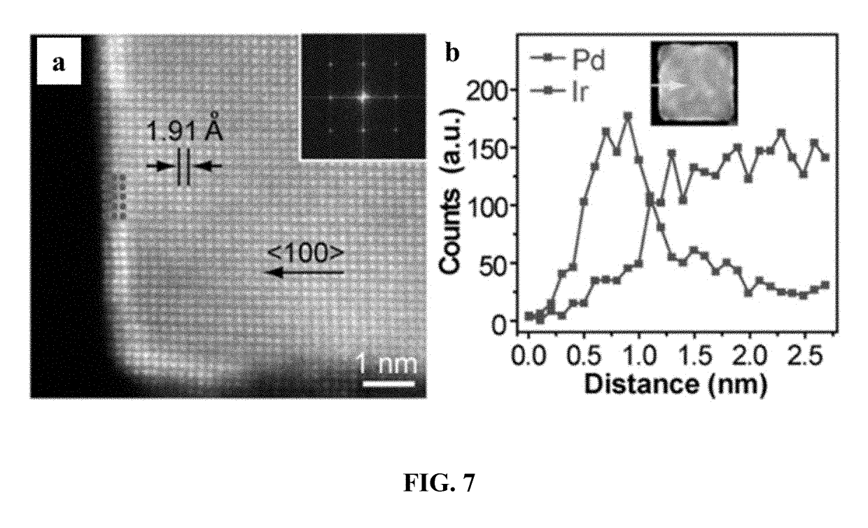

Characterization of Nanoparticles. The 18 nm Pd cubic seeds (FIG. 4) were prepared as described above. FIG. 6a shows a typical TEM image of the Pd—Ir cubes prepared from a standard synthesis. It can be seen that the cubic shape of the Pd seeds was retained after the growth of Ir. Thin Ir shells (with darker contrast) over the Pd seeds can be resolved by a close examination owing to the difference in atomic number between Ir and Pd. The low-magnification TEM and high-angle annular dark-field scanning TEM (HAADF-STEM) images demonstrated that the Pd—Ir cubes could be obtained with a high quality. The analyses of 200 particles indicated that (i) 98.6% of particles displayed well-defined cubic shapes; (ii) the products had a narrow size distribution (1.31 nm standard deviation); and (iii) the average edge length of the Pd—Ir cubes was about 19.2 nm, which was about 1.2 nm greater than that of the initial Pd cubic seeds. Therefore, on average, the thickness of the Ir shell deposited on each side face of a Pd cubic seed was about 0.6 nm. FIG. 6b shows a HAADF-STEM image taken from an individual Pd—Ir cube, from which a thin Ir shell on the surface of the Pd seed can be clearly seen (with a brighter contrast). The enlarged atomic-resolution HAADF-STEM image together with its Fourier transform pattern (FIG. 7a) reveals the continuous lattice fringes from the Pd core to the Ir shell, indicating an epitaxial relationship between these two elements. FIG. 7a also shows that Ir atoms were unevenly coated on the surfaces of Pd seeds with a maximum number of atomic layers of 3, implying the involvement of 3D island growth mode during synthesis. FIG. 7b shows a line profile of energy-dispersive X-ray spectroscopy (EDX) analysis recorded from an individual Pd—Ir cube, further confirming the elemental compositions of the Ir shell and Pd core. The molar ratio of Ir to Pd (PdIrₓ) was quantified for these Pd—Ir cubes using inductively coupled plasma atomic emission spectroscopy (ICP-AES), which was measured to be PdIr_{0.062}. According to the ICP-AES data, the size of Pd cubic seeds, and the unit cell parameters of Ir the average number of Ir atomic layers was estimated to be 1 for this sample, indicating an overall monolayer coating of Ir on the Pd cubic seeds.

### Example 2

### Evaluation of Peroxidase-like Activity of Pd—Ir Nanoparticles

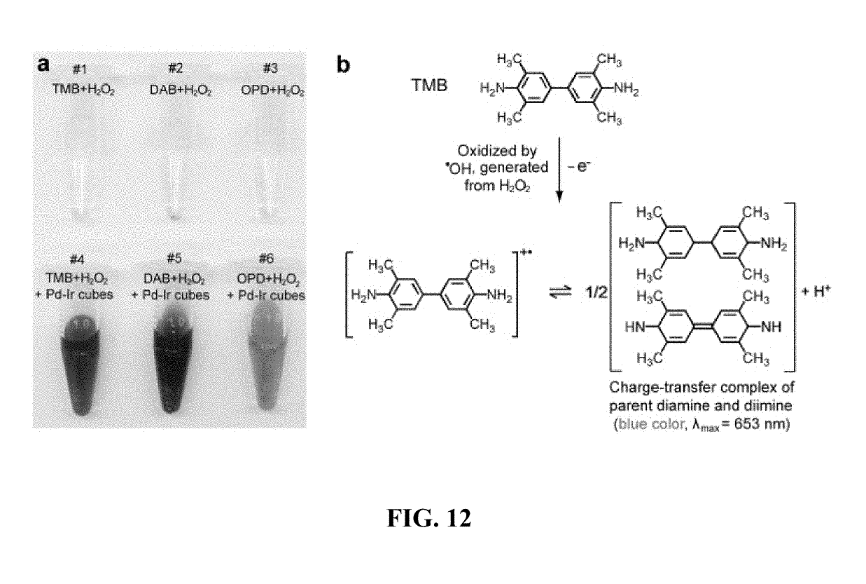

Methods. The experiments were conducted at room temperature in 1.5 mL tubes. For the oxidation of TMB by H₂O₂, the reactions were carried out in 1.0 mL of a 0.2 M NaOAc/HOAc buffer solution, pH 4.0, containing ˜5×10¹⁴M Pd—Ir cubes as the catalysts and 2 M H₂O₂ and 0.8 mM TMB as the substrate. The reaction buffers for OPD and DAB were 0.2 M Na₂HPO₄+0.1 M citric acid and 0.1 M Tris-HCl+0.15 M NaCl (pH 7.8), respectively. For the measurements in FIGS. 12 & 13, the absorbance at 653 nm of the reaction solutions containing 3.4×10¹⁴ M Pd—Ir cubes, TMB, and H₂O₂ in 0.2 M NaOAc/HOAc buffer at different conditions (pH, temperatures, TMB and H₂O₂ concentrations) was measured at t=5 min (reaction time was recorded immediately after the mixture of Pd—Ir cubes and substrates). The catalytic activity of Pd—Ir cubes was characterized by the relative reactivity with the maximal absorbance being set as 100%.

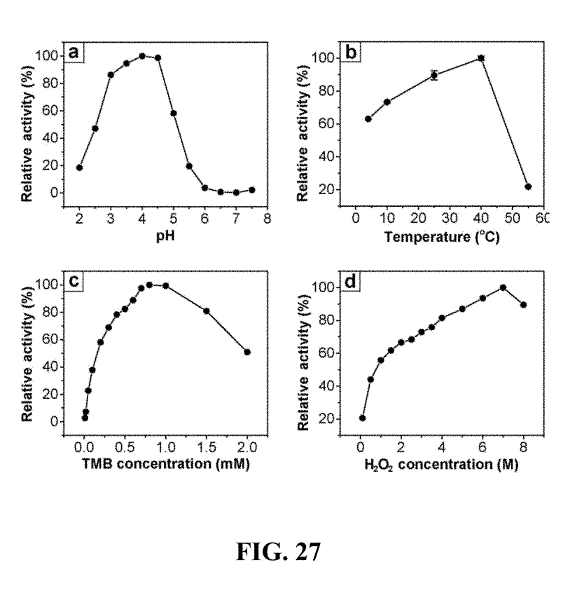

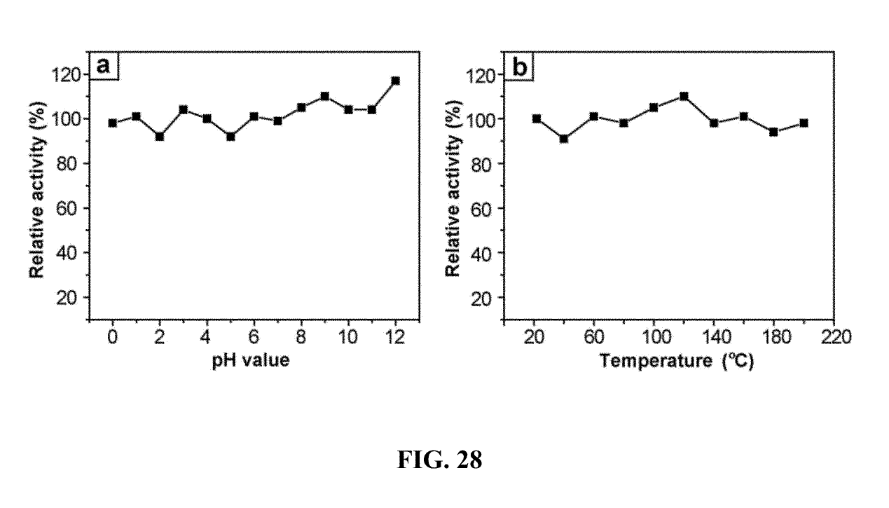

Results. As shown in FIGS. 12 & 13, the Pd—Ir cubes could rapidly catalyze the oxidation of 3,30,5,50 tetramethylbenzidine (TMB, a typical HRP substrate) by H₂O₂, yielding a blue-colored product with a maximum absorbance at 653 nm (FIGS. 12b & 13a). In addition to TMB, the Pd—Ir nanoparticles can also catalyze the oxidation of several other peroxidase substrates such as 3,30-diaminobenzidine (DAB) and o-phenylenediamine (OPD), producing brown- and orange-colored products, respectively (FIG. 12a). These results clearly demonstrated the peroxidase-like property of the Pd—Ir cubes. Using the oxidation of TMB by H₂O₂ as a model reaction, it was found that the catalytic efficiency of the Pd—Ir cubes (3.4×10⁻¹⁴ M particle concentration) is dependent on pH, temperature, and substrate and H₂O₂ concentrations. On the basis of the systematic examination of these factors (FIG. 27), exemplary conditions were found to be pH 4.0, 37 C., 0.8 mM TMB, and 7.0 M H₂O₂. It should be pointed out that no significant change of activity was observed for temperatures at 10-37 ° C. and H₂O₂ concentrations at 1.5-7.0 M. Therefore, for simplicity, a pH 4.0, room temperature (˜22 ° C.), 0.8 mM TMB, 2.0 M H₂O₂, and 3.4×10¹⁴M Pd—Ir cubes were used as the standard conditions for subsequent studies of the 19.2 nm PdIr cubes. As shown in FIG. 28(b), the catalytic activities of the Pd—Ir cubes remained stable after they had been treated with heat (40-200° C.) and acid or base (pH 0-12, FIG. 28(a)) for 2 h, indicating their outstanding thermal and chemical stabilities.

### Example 3

### Kinetic Analysis of Pd—Ir Nanoparticles

Methods. All steady-state kinetic assays were carried out at room temperature in 1.0 mL cuvettes with a path length (1) of 1.0 cm. Unless otherwise stated, a 0.2 M NaOAc/HOAc solution (pH 4.0) was used as the reaction buffer. Final concentrations of 1.4×10⁻¹²M for the 18 nm Pd cubes, 2.2×10⁻¹³M for the 8.4 nm Pd—Ir cubes, 3.4×10⁻¹⁴M for the 19.2 nm Pd—Ir cubes, and 4.8×10⁻¹⁵M for the 52.2 nm Pd—Ir cubes were used for their corresponding kinetic assay measurements. For the Pd—Ir cubes with different molar ratios of Ir to Pd, the final particle concentration was 3.4×10⁻¹⁴M for their kinetic assay measurements. After addition of substrates (TMB and H₂O₂) in the buffer system containing cubes, the absorbance of the reaction solution at 653 nm of each sample was immediately measured as a function of time with intervals of 6 s using a spectrophotometer for 3 min. These “absorbance vs time” plots were then used to obtain the slope at the initial point (Slope_{Initial}) of each reaction by conducting the first derivation of each curve using OriginPro 9.0 software. The initial reaction velocity (v) was calculated by Slope_{Initial}/(ε_{TMB-653 nm}×1), where ε_{TMB-653 nm} is the molar extinction coefficient of TMB at 653 nm, which equals 3.9×10⁻¹⁴ M·cm⁻¹. The plots of v against substrate concentrations were fitted using nonlinear regression of the Michaelis-Menten equation. The apparent kinetic parameters were calculated based on the Michaelis-Menten equation v=Vₘₐₓ×[S]/(Kₘ+[S]), where Vₘₐₓ represents the maximal reaction velocity, [S] is the concentration of substrate, and Kₘ is the Michaelis constant. Parameters Kₘ and Vₘₐₓ were obtained from the double reciprocal plot (or Lineweavere-Burk plot).

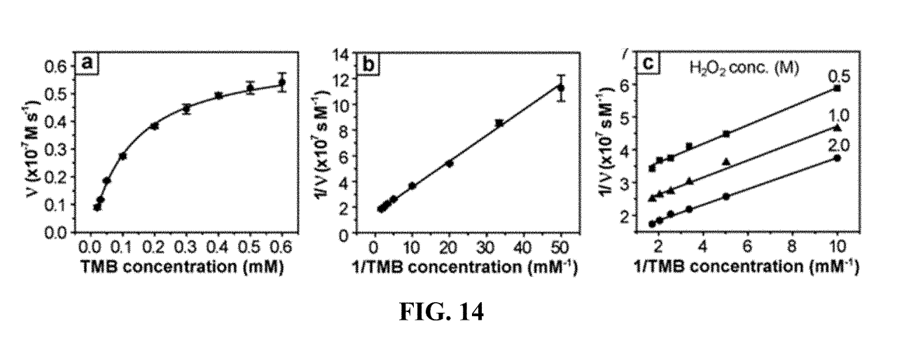

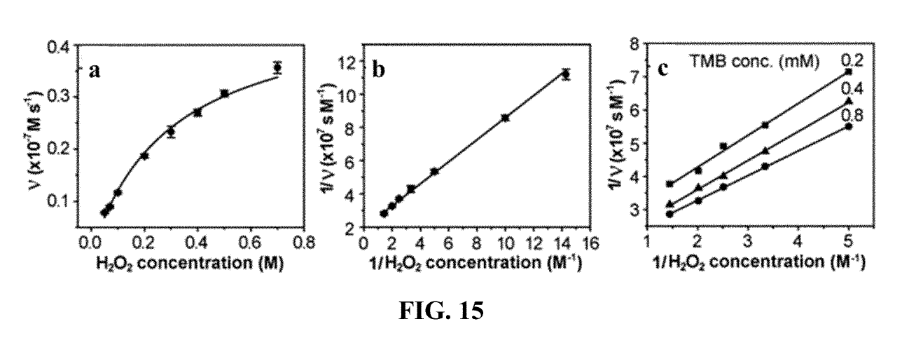

Results. In order to quantify the catalytic efficiency and understand the catalytic mechanism of the Pd—Ir cubes, the apparent steady-state kinetic parameters for the reaction between TMB and H₂O₂were evaluated. By plotting the initial reaction velocities against substrate concentrations, typical Michealis-Menten curves were observed for both TMB (FIG. 14a) and H₂O₂ (FIG. 15a). The curves were then fitted to the double-reciprocal or Lineweaver Burk plots (FIG. 14b & 15b) from which the kinetic parameters shown in Table 1 were determined. For comparison, the kinetic parameters of 18 nm Pd cubic seeds were also determined and listed those of HRP (Table 1). It can be seen that the K_{cat} value, which measures the efficiency of a catalyst, of the 19.2 nm Pd—Ir cubes is more than 20- and 400-fold higher than those of 18 nm Pd cubic seeds and HRP, respectively. The Kₘ value of the Pd—Ir cubes was ˜100-fold higher than that of HRP toward H₂O₂, suggesting that Pd—Ir cubes had a lower affinity for H₂O₂ than HRP. FIGS. 14c & 15c show the double-reciprocal plots at different H₂O₂ and TMB concentrations, respectively. The lines in each plot are parallel, which is characteristic of a typical ping-pong mechanism as was observed for HRP. This result indicated that, like HRP, the Pd—Ir cubes bind and react with one substrate (either TMB or H₂O₂) and then release a product before reacting with the other substrate.

TABLE 1
list of kinetic parameters of various Pd—Ir cubes as peroxidase
mimics toward the oxidation of TMB by H₂O₂. [E] represents the
mimic concentration, Kₘ is the Michaelis constant, Vₘₐₓ is the maximal
reaction velocity, K_{cat} is the catalytic constant that equals Vₘₐₓ/[E].

| | | | Kₘ | Vₘₐₓ | K_{cat} |
|---|---|---|---|---|---|
| Catalyst | [E] (M) | Substance | (M) | (M s⁻¹) | (s⁻¹) |
| 19.2 nm | 3.4 × 10⁻¹⁴ | TMB | 1.3 × 10⁻⁴ | 6.5 × 10⁻⁸ | 1.9 × 10⁶ |
| PdIr cubes | 3.4 × 10⁻¹⁴ | H₂O₂ | 3.4 × 10⁻¹ | 5.1 × 10⁻⁸ | 1.5 × 10⁶ |
| 18 nm | 1.4 × 10⁻¹² | TMB | 5.4 × 10⁻⁵ | 9.7 × 10⁻⁸ | 6.9 × 10⁴ |
| Pd cubes | 1.4 × 10⁻¹² | H₂O₂ | 7.0 × 10⁻¹ | 6.5 × 10⁻⁸ | 4.6 × 10⁴ |
| HRP | 2.5 × 10⁻¹¹ | TMB | 4.3 × 10⁻⁴ | 1.0 × 10⁻⁷ | 4.0 × 10³ |
| | 2.5 × 10⁻¹¹ | H₂O₂ | 3.7 × 10⁻³ | 8.7 × 10⁻⁸ | 3.5 × 10³ |

### Example 4

### Effect of Ir Content

Methods. Methods of preparing Pd—Ir nanoparticles and catalytic efficiency were performed as described above.

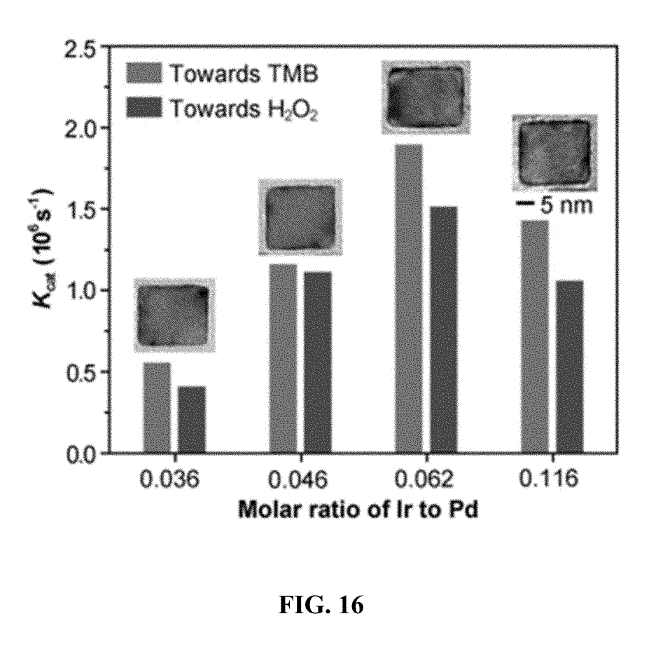

Results. Interestingly, the enhanced activity of Pd—Ir cubes relative to the initial Pd seeds can be attributed to the coat of Ir shells. To better understand the role played by Ir shells in determining the catalytic efficiency, a set of Pd—Ir cubes was prepared with different molar ratios of Ir to Pd (PdIrₓ) from the same batch of Pd seeds by adjusting the amount of Ir precursor introduced to the reaction solution and then evaluated their efficiencies. FIG. 16 shows representative TEM images of these samples and comparison of their K_{cat} values. A thin Ir shell (with a darker contrast) over the Pd seed can be resolved from the TEM images, in which thickness gradually increases with Ir content. TEM images at a lower magnification suggest good uniformities of all these samples. The Kcat values of the four different PdIrₓ samples (x=0.036, 0.046, 0.062, and 0.116) toward both TMB and H₂O₂ showed a volcano shaped dependence on the Ir contents, with maximum points corresponding to PdIr_{0.062} which are around 4 times those of the PdIr_{0.036}. Deposition of more than 6.2% of Ir on the Pd seeds leads to a decrease in K_{cat} values.

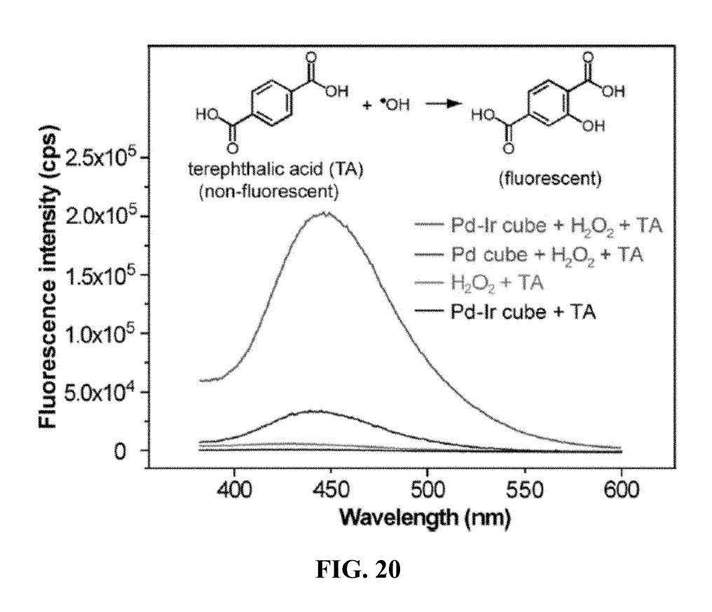

Although the explicit mechanism of the peroxidase mimics-mediated catalytic reaction is not known, generation of hydroxyl radicals (·OH) from H₂O₂ and the oxidation of TMB by ·OH to form the blue-colored complex were believed to be two important steps of this reaction. To understand why the Ir-coated Pd cubes showed a better catalytic performance than the pristine Pd cubes, their efficiencies were compared in generating ·OH from H₂O₂ using terephthalic acid (TA) as a probe, which could easily react with ·OH to form a highly fluorescent 2-hydroxyterephthalic acid (FIG. 20). In a typical procedure, 0.5 mM TA, 0.2 M H₂O₂, and ˜2×10¹⁰ particles/mL 19.2 nm Pd—Ir cubes or 18 nm Pd cubes were incubated in 5 mL of 0.2 M NaOAc/HOAc buffer (pH 4.0) for 20 min at room temperature, followed by fluorescence measurements using a spectrofluorometer. As shown in FIG. 20, intense fluorescence was detected for solutions containing nanoparticles, H₂O₂, and TA, while very weak fluorescence signal was detected in the absence of either H₂O₂ or nanoparticles. This observation demonstrated that, for the present system, most of the ·OH species were generated from H₂O₂ in the presence of nanoparticles as catalysts. Interestingly, the fluorescence intensity of the solution of Pd—Ir cubes, H₂O₂, and TA was much stronger than that of the solution of Pd cubes, H₂O₂, and TA. This result indicated that the Pd—Ir cubes were more efficient in generating ·OH from H₂O₂ than Pd cubes, which may be responsible for their superior catalytic performance toward the oxidation of TMB by H₂O₂ relative to Pd cubes.

### Example 5

### Size Control of Pd—Ir Nanoparticles

Methods. Methods of preparing Pd—Ir nanoparticles and catalytic efficiency were performed as described above.

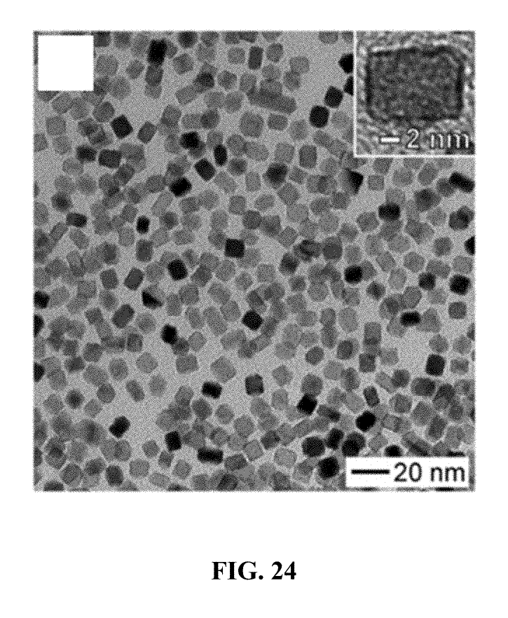

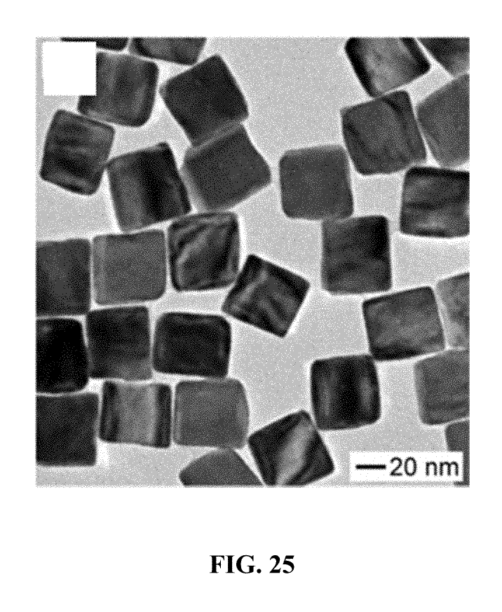

Results. It should be noted that the size of the Pd—Ir cubes can be readily controlled by coating Pd cubic seeds of different sizes with ˜0.6 nm Ir shells. For instance, sub-10-nm Pd—Ir cubes with an average edge length of 8.4 nm (see FIG. 24) can be obtained by using Pd cubes of 7.2 nm in edge length as the seeds. In addition, 52.2 nm Pd—Ir cubes (FIG. 25) can be prepared from Pd cubes of 51 nm in edge length as the seeds. TEM images taken from these products indicate that Pd—Ir cubes can be synthesized in high purity and good uniformity regardless of particle size. The K_{cat} values of the 8.4, 19.2, and 52.2nm Pd—Ir cubes were calculated to be roughly at levels of 105, 106 , and 107 s⁻¹, respectively. This increase of K_{cat} with particle size may be ascribed to the fact that larger cubes have a greater surface area to interact with substrates. In this regard, the three Pd—Ir cubes showed similar specific efficiencies (defined as the normalized K_{cat} to the surface area of an individual catalyst, K_{cat}/S) at a level of 102 ^{s-1} nm⁻² (Table S2).

It is worth noting that the molar ratios of Ir to Pd for the 8.4 and 52.2 nm Pd—Ir cubes were determined to be 0.191 and 0.022, respectively, which correspond to approximate single Ir overlayers on both the 7.2 and 51 nm Pd cubic seeds. Deposition of less or more than 19.1% of Ir on the 7.2 nm Pd seeds led to a decrease in their catalytic activities. A similar trend was also observed for the 51 nm Pd seeds.

TABLE 2
Kinetic Analysis of Different Pd—Ir Cube Sizes

| | | | Kₘ | Vₘₐₓ | K_{cat} | K_{cat}/S^{a} |
|---|---|---|---|---|---|---|
| Catalyst | [E] (M) | Substance | (M) | (M s⁻¹) | (s⁻¹) | (s⁻¹nm⁻²) |
| 8.4 nm | 2.2 × 10⁻¹³ | TMB | 1.2 × 10⁻⁴ | 8.5 × 10⁻⁸ | 3.9 × 10⁵ | 9.2 × 10² |
| Pd—Ir cubes | | | | | | |
| 19.2 nm | 3.4 × 10⁻¹⁴ | TMB | 1.3 × 10⁻⁴ | 6.5 × 10⁻⁸ | 1.9 × 10⁶ | 8.6 × 10² |
| Pd—Ir cubes | | | | | | |
| 52.2 nm | 4.8 × 10⁻¹⁵ | TMB | 1.9 × 10⁻⁴ | 5.5 × 10⁻⁸ | 1.2 × 10⁷ | 7.3 × 10² |
| Pd—Ir cubes | | | | | | |

Table 2 lists kinetic parameters of various Pd—Ir cubes as peroxidase mimics toward the oxidation of TMB by H₂O₂. [E] represents the mimic concentration, Kₘ is the Michaelis constant, Vₘₐₓ is the maximal reaction velocity, K_{cat} is the catalytic constant that equals Vₘₐₓ/[E]. K_{cat}-specific represents the specific catalytic efficiency that is derived by normalizing K_{cat} against the surface area of an individual mimic. ^{a} Surface area (S) was estimated based on the hypothesis that the particles are ideal cubes with uniform sizes.

Kinetic parameters were also investigated for a Pd—Ir octahedral embodiment, and these results are laid out below in Table 3.

TABLE 3
Kinetic Analysis of a Pd—Ir Octahedral Embodiment

| | | | Kₘ | Vₘₐₓ | K_{cat} | K_{cat}/S^{a} |
|---|---|---|---|---|---|---|
| Catalyst | [E] (M) | Substance | (M) | (M s⁻¹) | (s⁻¹) | (s⁻¹nm⁻²) |
| 6.1 nm | 1.5 × 10⁻¹² | TMB | 2.0 × 10⁻⁴ | 1.7 × 10⁻⁷ | 1.1 × 10⁵ | 8.8 × 10² |
| Pd—Ir octahedra | | | | | | |

Table 2 lists kinetic parameters of 6.1 nm Pd—Ir octahedra toward the oxidation of TMB by H₂O₂. [E] represents the mimic concentration, Vₘₐₓ is the maximal reaction velocity, K_{cat} is the catalytic constant that equals Vₘₐₓ/[E]. K_{cat}specific represents the specific catalytic efficiency that is derived by normalizing K_{cat} against the surface area of an individual mimic. ^{a} Surface area (S) was estimated based on the hypothesis that the particles are ideal octahedra with uniform sizes.

Kinetic parameters were also investigated for various Pt cubes as a function of capping agent molecular weight, and these results are laid out below in Table 4.

TABLE 4
Kinetic Analysis of Different Pt—Ir Cube Sizes

| | | | Kₘ | Vₘₐₓ | K_{cat} | K_{cat}/S^{a} |
|---|---|---|---|---|---|---|
| Catalyst | [E] (M) | Substance | (M) | (M s⁻¹) | (s⁻¹) | (s⁻¹nm⁻²) |
| 7.35 nm | 4.1 × 10⁻¹³ | TMB | 7.3 × 10⁻⁴ | 3.3 × 10⁻⁷ | 8.2 × 10⁵ | 2.5 × 10³ |
| PVP₅₅-Pt cubes^{b} | | | | | | |
| 6.69 nm | 5.4 × 10⁻¹³ | TMB | 5.2 × 10⁻⁴ | 2.4 × 10⁻⁷ | 4.4 × 10⁵ | 1.6 × 10³ |
| PVP₁₀-Pt cubes^{c} | | | | | | |
| 8.24 nm | 2.6 × 10⁻¹³ | TMB | 8.1 × 10⁻⁴ | 3.0 × 10⁻⁷ | 1.2 × 10⁶ | 2.8 × 10³ |
| PVP₃₆₀-Pt cubes^{d} | | | | | | |

Comparison of kinetic parameters of various Pt cubes as peroxidase mimics toward the oxidation of TMB by H₂O₂. [E] represents the mimic concentration, Kₘ is the Michaelis constant, Vₘₐₓ is the maximal reaction velocity, K_{cat} is the catalytic constant that equals Vₘₐₓ/[E]. K_{cat}-specific represents the specific catalytic efficiency that is derived by normalizing K_{cat} against the surface area of an individual mimic. ^{a}Surface area (S) was estimated based on the hypothesis that the particles are ideal cubes with uniform sizes. ^{b}PVP₅₅ refers to the polyvinylpyrrolidone with molecular weight (Mw) of 55,000. ^{c}PVP₁₀ refers to the polyvinylpyrrolidone with molecular weight (Mw) of 10,000. ^{d}PVP₃₆₀ refers to the polyvinylpyrrolidone with molecular weight (Mw) of 360,000. From this table, it appears that increased molecular weight of the capping agent improves catalytic efficiency.

### Example 6

### PEGylation of Pd—Ir Nanoparticles

Methods. The method for PEGylation of 19.2 nm Pd—Ir Cubes and quantification of PEG chains on the Pd—Ir Cube. In a typical PEGylation process, 300 μL of an aqueous HS-PEG3400-COOH or HS-PEG3400-NH₂ solution at a specific concentration was added to 300 μL of an aqueous suspension of 19.2 nm Pd—Ir cubes (8.5×10⁻⁸M). The mixture was incubated at room temperature with stirring for 3 h, followed by centrifuging at 14 000 rpm for 10 min. The supernatant was carefully collected and centrifuged two more times to remove the remaining PdIr cubes. On the other hand, the precipitates (i.e., Pd—Ir cube-S-PEG3400-COOH or Pd—Ir cube-SPEG3400-NH₂) were washed three times with DI water and redispersed in 300 μL of DI water for future use. The average number of PEG chains on each Pd—Ir cube could be determined using the Ellman test, in which thiol groups in PEG chains react with Ellman's reagent (i.e., 5,50-dithiobis(2-nitrobenzoic acid), DTNB). The same method could be used to PEGylation of other Pd—Ir nanostructures.

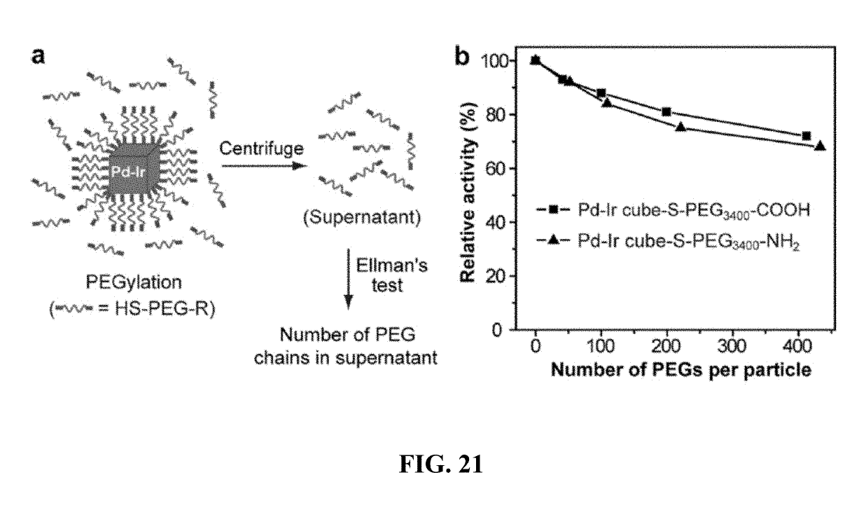

Results. Taking advantage of the Ir-thiolate bonding, the surface of the Pd—Ir cubes were modified with various functional groups such as COOH and NH₂ through the PEGylation of thiolated heterobifunctional polyethylene glycol (HS-PEG-R) chains (FIG. 21). These functional groups on particle surfaces can serve as reactive sites to link various ligands such as peptides, nucleic acids, and antibodies. Interestingly, the average number of PEG chains on each Pd—Ir cube can be conveniently quantified by subtracting the number of free PEG chains left in the supernatant from the total number of PEG chains added to the Pd—Ir cube suspension during PEGylation (FIG. 21(a)). Using Pd—Ir cube-S-PEG₃₄₀₀-COOH and Pd—Ir cube-S-PEG₃₄₀₀-NH₂ as model examples, it was found that the catalytic activity of Pd—Ir cubes was well retained after they had been PEGylated with various numbers of PEG chains (FIG. 21(b)). For instance, on average, the catalytic activity of a Pd—Ir cube decreased by only ˜17% after it had been loaded with 200 -S-PEG₃₄₀₀-NH₂ chains. It is worth noting that the maximum number of -S-PEG₃₄₀₀-COOH and -S-PEG₃₄₀₀-NH₂ chains loaded onto an individual Pd—Ir cube were determined to be ˜3300 and ˜4100, respectively. These results indicate the simplicity and versatility of the Pd—Ir cubes in labeling ligands.

### Example 7

### Pd—Ir-based ELISA

Methods. The method for preparation of the Pd—Ir cube-goat anti-mouse IgG conjugates. The Pd—Ir cube-goat anti-mouse IgG conjugates were prepared by labeling the Pd—Ir cube-S-PEG₃₄₀₀-COOH (˜200 -SPEG₃₄₀₀-COOH chains per cube) with anti-mouse IgG using EDC and NHS as coupling agents. In brief, 50 μL of Pd—Ir cube-S-PEG₃₄₀₀-COOH (8.5×10⁻⁸ M, in DI water) was added to 450 μL of a 10 mM phosphate-buffered saline (PBS, pH 7.4) buffer at room temperature under stirring. Then, 5 μL of EDC (25 mM, in DI water) and 5 μL of NHS (50 mM, in DI water) were added. After 15 min, the particles were washed with DI water twice and redispersed in 50 μL of PBS. Subsequently, 50 μL of goat anti-mouse IgG (2 mg/mL, in PBS) was added to the particle suspension. After incubation at room temperature for 1 h, the reaction solution was put in a refrigerator overnight at 4° C. Thereafter, 100 μL of blocking solution (5% BSA in PBS) was added to the reaction solution. After 2 h, the final products were collected by centrifugation, washed twice with PBS, and redispersed in 100 μL of PBS containing 1% BSA and 0.05% NaN₃. The same method could be used to prepare the conjugates of other Pd—Ir nanostructures.

Pd—Ir ELISA of PSA. The 96-well microtiter plates were coated with rabbit anti-PSA pAb (100 μL, 5 μg/mL in carbonate/bicarbonate buffer pH 9.6) at 4° C. overnight. After washing the plates three times with washing buffer (10 mM PBS pH 7.4 containing 0.5% Tween 20, PBST), the plates were blocked with 200 μL of blocking buffer (3% BSA in PBST) for 3 h at room temperature. The plates were then washed three times with washing buffer, followed by the addition of 100 μL of PSA standards in dilution buffer (1% BSA in PBST). After shaking at room temperature for 2 h, the plates were washed three times with washing buffer, and 100 μL of mouse anti-PSA mAb (2 μg/mL, in dilution buffer) was added. After 1 h shaking at room temperature, the plates were washed three times, and 100 μL of Pd—Ir cube-goat anti-mouse IgG conjugates (1:5000, in dilution buffer) was added, followed by 30 min shaking at room temperature. After washing four times, 100 mL of freshly prepared substrate solution (0.8 mM TMB and 2.0 M H₂O₂ in 0.2 M HOAc/NaOAc buffer, pH 4.0) was added. After 30 min incubation at room temperature, 50 μL of stop solution (2 M H₂SO₄) was added. The absorbance of each well at 450 nm was read using a microplate reader. The procedure of HRP ELISA was the same as the Pd—Ir ELISA except for the substitutions of Pd—Ir cube-goat antimouse IgG conjugates with 100 μL of HRP-goat anti-mouse IgG conjugates (1 μg/mL, in dilution buffer) and the difference in the components of substrate solution (0.8 mM TMB and 5 mM H₂O₂ in citric acid/Na₂HPO₄ buffer, pH 5.0). The same method could be used to perform the ELISA of PSA for the other Pd—Ir nanostructures.

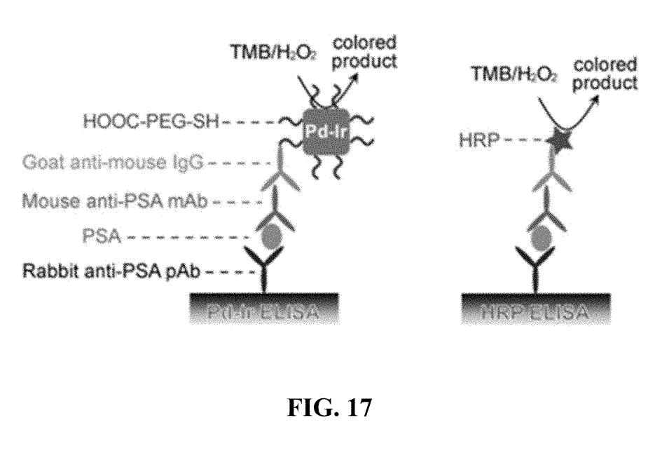

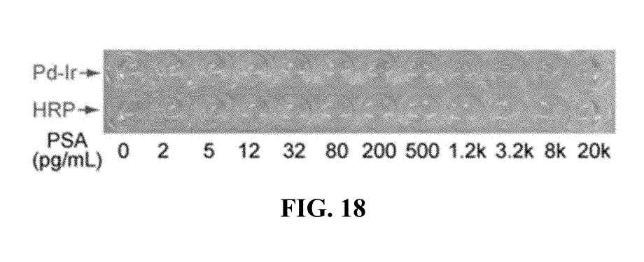

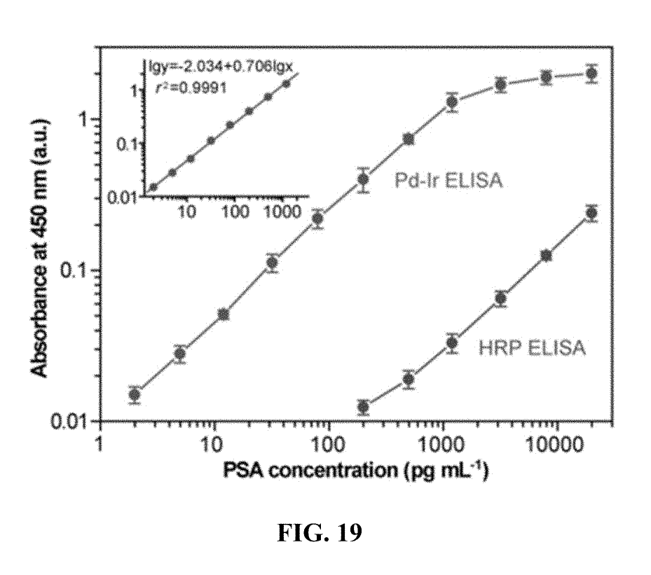

Results. In this example, Pd—Ir cubes were applied to a colorimetric enzyme-linked immunosorbent assay (ELISA) of human PSA, a prostate cancer biomarker. As shown in FIG. 17, the principle of the Pd—Ir ELISA is similar to the conventional colorimetric HRP ELISA except for the substitution of HRP with Pd—Ir cubes. Here, the Pd—Ir cube-goat anti-mouse IgG conjugates were prepared from the 19.2 nm Pd—Ir cubes. In brief, the Pd—Ir cubes were first PEGylated with HS-PEG₃₄₀₀-COOH. The resultant Pd—Ir cube-SPEG₃₄₀₀-COOH were then used to conjugate goat anti-mouse IgG through the EDC/NHS-mediated coupling reaction between the carboxyl groups on cubes and the primary amino groups on the antibodies. For head-to-head comparison, the Pd—Ir ELISA was compared against the conventional HRP ELISA by using the same set of antibodies and PSA standards. The procedures of both ELISAs were also kept the same. PSA standards with various concentrations were monitored in a 96-well microtiter plate (FIG. 18) and quantified using a plate reader, where the yellow-colored products (i.e., diimine, λmax 450 nm) were generated by quenching the catalytic reaction using H₂SO₄. A sigmoid curve regression between the logarithms of absorbance and PSA concentration was obtained for the Pd—Ir ELISA (FIG. 19). A quality linear relationship (r²=0.9991) in the range 2-1200 pg/mL PSA was observed. The coefficient of variations across the entire concentration range was between 1.7% and 11.8% (n=6), indicating good reproducibility. The detection limit, defined as the concentration corresponding to a signal that is 3 times the standard deviation above the zero calibrator, was calculated to be 0.67 pg/mL. In comparison, the detection limit of the HRP ELISA was determined to be 75 pg/mL based on its calibration curve (FIG. 19), which is approximately 110-fold higher than that of Pd—Ir ELISA. This significant increase in detection sensitivity for the Pd—Ir ELISA is hypothesized to be attributed to the much higher catalytic efficiency of the Pd—Ir cubes relative to HRP because other conditions of both ELISAs were kept identical. The batch-to-batch reproducibility of the Pd—Ir ELISA was also evaluated by assaying a 50 pg/mL PSA standard using six different batches of Pd—Ir cubes (three duplicates for each batch), in which the coefficient of variation of the average absorbance at 450 nm was calculated to be 16.3%. These results suggest that the Pd—Ir ELISA is a promising candidate for detection of disease biomarkers, which has a high sensitivity rivaling the limits of fluorescent and plasmonic methods while retaining the simplicity and reliability of conventional colorimetric ELISA.

What is claimed is:

1. A method of catalyzing the oxidation of a substrate by an oxidizing agent, the method comprising contacting the substrate with the oxidizing agent and a nanoparticle in a reaction mixture, wherein the nanoparticle comprises a Pd core at least partially surrounded by a coating comprising Ir.

2. The method of claim 1, wherein the nanoparticle has a shape selected from the group consisting of cubic, truncated cubic, octahedral, truncated octahedral, and spherical.

3. The method of claim 1, wherein the nanoparticle comprises Pd and Ir at a molar ratio of from about PdIr_{0.01} to about PdIr_{0.3}.

4. The method of claim 1, wherein the nanoparticle further comprises a capping agent.

5. The method of claim 4, wherein the capping agent is polyvinylpyrrolidone.

6. The method of claim 1, wherein the Pd core has an average surface area of from about 25 nm² to about 2.5×10⁵ nm².

7. The method of claim 1, wherein the Pd core comprises {100} facets, {111} facets, or a combination thereof.

8. The method of claim 1, wherein the coating has an average thickness of from about 0.2 nm to about 2 nm.

9. The method of claim 1, wherein the coating has a thickness of from about 1 atomic layer to about 3 atomic layers.

10. The method of claim 1, wherein the coating comprises Ir {100} facets, Ir {111} facets, or a combination thereof.

11. The method of claim 1, wherein the oxidizing reagent is present in the reaction mixture at a concentration of from about 0.1 M to about 20 M.

12. The method of claim 1, wherein the oxidizing agent is hydrogen peroxide.

13. The method of claim 1, wherein the substrate is present in the reaction mixture at a concentration of from about 0.1 nM to about 10 M.

14. The method of claim 1, wherein the reaction mixture has a temperature of from about 10° C. to about 50° C.

15. The method of claim 1, wherein the reaction mixture has a pH of from about 2 to about 8.

16. The method of claim 1, wherein the substrate is chromogenic, chemiluminescent, fluorogenic, electrochemiluminescent or a combination thereof.

17. The method of claim 16, wherein the substrate is selected from the group consisting of 2,T-azino-bis(3-ethylbenzthiazoline-6-sulfonic acid) (ABTS), 3,3′,5,5′-tetramethylbenzidine (TMB), 3,3′,5,5′-tetramethylbenzidine sulfate (TMBS), o-phenylenediamine (OPD), o-phenylenediamine dihydrochloride (OPD-2HCl), diaminobenzidine (DAB), diaminobenzidine tetrahydrochloride (DAB-4HCl), 3-amino-9-ethylcarbazole (AEC), 10-acetyl-3,7-dihydroxyphenoxazine (Ampliflu Red), luminol, homovanillic acid, 5-aminosalicylic acid (5-ASA), and a combination thereof.

18. The method of claim 1, wherein the nanoparticle is coupled to a binding agent adapted to bind to a target analyte, and wherein the method further comprises contacting the nanoparticle and the binding agent with the target analyte to form a complex comprising the nanoparticle, the binding agent and the target analyte, and contacting the complex with the substrate and the oxidizing agent in the reaction mixture.

19. The method of claim 18, further comprising detecting or quantifying the target analyte in the reaction mixture.

20. The method of claim 1, wherein the substrate is coupled to a binding agent adapted to bind to a target analyte, and wherein the method further comprises contacting the substrate and the binding agent with the target analyte to form a complex comprising the substrate, the binding agent and the target analyte, and contacting the complex with the nanoparticle and the oxidizing agent in the reaction mixture.

21. The method of claim 20, further comprising detecting or quantifying the target analyte in the reaction mixture.

22. A kit for catalyzing the oxidation of a substrate by an oxidizing agent, the kit comprising:
the substrate
the oxidizing agent; and
a nanoparticle, wherein the nanoparticle comprises a Pd core at least partially surrounded by a coating comprising Ir; and
instructions describing how to contact the substrate, the oxidizing agent and the nanoparticle so as to catalyze the oxidation of the substrate.

23. The kit of claim 22, wherein the nanoparticle or the substrate is coupled to a binding agent adapted to specifically bind to a target analyte.
